(19) United States
(12) Patent Application Publication
(10) Pub. No.: US 2025/0120742 A1
(43) Pub. Date: Apr. 17, 2025

(54) RETENTION ASSEMBLY, SHAFT SEAL ASSEMBLY, AND SURGICAL INSTRUMENT

(71) Applicant: FlexDex, Inc., Brighton, MI (US)

(72) Inventors: Christopher Paul Huang Shu, Ann Arbor, MI (US); Zachary R. Zimmerman, Brighton, MI (US); Matthew P. Weber, Brighton, MI (US); Dustin Fichter, South Lyon, MI (US); James Duncan Geiger, Ottawa Hills, OH (US); Rogelio Insignares, Howell, MI (US)

(21) Appl. No.: 18/917,946

(22) Filed: Oct. 16, 2024

Related U.S. Application Data
(60) Provisional application No. 63/544,351, filed on Oct. 16, 2023.

(51) Int. Cl. A61B 17/34; A61B 17/00

(52) U.S. Cl. CPC A61B 17/3468; A61B 2017/0046

(57) ABSTRACT

A retention assembly is provided that can be equipped in a handheld surgical tool or instrument or other medical device, as well as potentially in non-medical devices, among other possible applications. In the application of the handheld surgical instrument, the retention assembly furnishes partial or more constraint between bodies such as a handle body and a frame body, which in turn serves to partially or more constrain an end effector of the handheld surgical instrument. Such constraint at the end effector, whether partial or full, can be useful in certain scenarios amid a minimally invasive surgical (MIS) procedure such as during initial insertion of the end effector through a surgical trocar at a patient's body.

### CROSS-REFERENCE TO RELATED APPLICATION

This application claims the benefit of U.S. Provisional Patent Application No. 63/544,351, with a filing date of Oct. 16, 2023, the contents of which are hereby incorporated by reference in their entirety.

### TECHNICAL FIELD

The present disclosure relates generally to articulating remote access tools, for example handheld surgical instruments that can be employed for use in minimally invasive surgical (MIS) procedures.

### BACKGROUND

Surgical tools are often designed and constructed with various components to have certain kinematic architectures at the handle and frame, and to ultimately furnish certain functionalities and performances at an end effector. Drawbacks can arise among the architectures depending on how the handle and frame and components are arranged and configured with respect to one another.

A variety of remote access tools have articulating joints at the distal end of a working device. The user controlled articulating joints may be comprised of one or more bodies connected by joints with rotational degrees of freedom. Attached to the articulating joint is a tool that is manipulated by the user. The user inputs are conveyed from a proximal input body (e.g., handle) to the articulation joint via a transmission system comprised of tension members, linkages, motors, or other means that transmit motions. In some use cases there is a need to immobilize the articulating joint and maintain the distal end of the joint in a fixed position.

For example, in the field of minimally invasive surgery (MIS), trained surgeons or their surgical assistants often use handheld articulating surgical devices to perform complex procedures. In these procedures a surgeon manipulates a surgical tool is attached to the articulating joint. With the surgical tool, the surgeon is able to perform complex procedures with precise motions such as accurately driving needles through tough tissue, grasping delicate tissue, dissecting tissue, retracting tissue, cutting tissue, fusing or stapling tissue together, tying knots with sutures, or cauterizing tissue. During these procedures devices are prepared and handed to a surgeon by a circulating or scrub nurse. The surgeon then grasps the user input of the device and inserts the device into the patient through a keyhole incision. Often, the surgeon utilizes a surgical trocar, or port located at the keyhole incision to serve as a guide for the surgical instrument insertion into the patient's body. This is a challenging step for the surgeon because they cannot accurately control the position and orientation of the end effector while only holding the user input. Surgeons have developed techniques to address the challenge, but they are often inefficient and require assistance. For example, the surgeon will use their opposite hand to provide additional support for the device or they will enlist the assistance of a nurse to support the device frame while the surgeon grasps the handle. This additional action is not only inefficient, but also may not be possible as there may not be any immediately free hands available to assist the surgeon; thus, resulting in inconvenience and surgical delays.

In scenarios where the surgeon is inserting a device into a trocar it is helpful to have a feature that allows the surgeon to control the position of the end-effector using only the input body. Some articulating devices do incorporate a feature that locks the position of the output body into a position relative to the user interface, but there are disadvantages to such systems. For example, U.S. Pat. No. 11,478,263 introduces a locking apparatus. The apparatus includes a user-controlled input member to engage a locking state that involves a pair of locking units that act on a set of articulation control members. Furthermore, a pair of unlocking units act upon the locking units to disengage the lock feature. While this apparatus locks the output body in a position relative to the input body, the functionality is achieved at the expense of necessitating a complex system with indirect locking functionality (immobilizes control members rather than rigid bodies).

Additionally, handheld articulating surgical instruments may be used to retract, or separate and hold tissues away from the surgeon's field of view during surgery. In these cases, and to case the effort to retract tissue, it can be helpful to lock and maintain the device in a desired articulated position.

Further, a variety of remote access tools have articulating joints at the distal end of a working device. The user controlled articulating joints may be comprised of one or more bodies connected by joints with rotational degrees of freedom. Attached to the articulating joint is a tool that is manipulated by the user. The user inputs are conveyed from a proximal input body (e.g., handle) to the articulation joint via a transmission system comprised of tension members, linkages, motors, or other means that transmit motions. In some use cases there is a need to isolate and maintain a pressure differential between the distal portion of the device and the proximal portion of the device during minimally invasive surgical procedures.

### SUMMARY

In an embodiment, a retention assembly may include a first body, a second body, a third body, one or more joints, and a retention body. The joint(s) is situated between the first body and the second body, and provides one or more degrees of freedom (DOF) between the first body and the second body. The retention body can be moved between a first position and a second position. When the retention body is in the second position, the retention body partially or more constrains at least one of the degree(s) of freedom between the first body and the second body, whereby the partial or more constraints is further effected at the third body. When the retention body is in the first position, the partial or more constraint between the first body and the second body by way of the retention body is absent, and the partial or more constraint at the third body is absent.

In another embodiment, a retention assembly may include a first body, a second body, one or more joints, and a retention body. The joint(s) is situated between the first body and the second body. The joint(s) provides a first degree of freedom between the first body and the second body, and provides a second degree of freedom between the first body and the second body. The retention body has one or more positional states. The positional state(s) partially or more and concurrently constrains the first degree of freedom and the second degree of freedom between the first body and the second body. The positional state(s) partially or more constrain movement of an end effector of the retention assembly.

In another embodiment, a method of partially or more constraining one or more degrees of freedom between bodies. The method involves moving a retention body from a first position to a second position. When the retention body is in the second position, one or more degrees of freedom between a first body and a second body is partially or more constrained. Further, when the retention body is in the first position, the partial or more constraint of the degree(s) of freedom between the first body and the second body is absent.

In another embodiment, a retention assembly may comprise a first body, a second body, one or more joints, and a magnet body. The joint(s) is situated between the first body and the second body. The joint(s) provides one or more degrees of freedom between the first body and the second body. The magnet body is carried by the first body or is carried by the second body. When the first body and the second body exhibit one or more positions with respect to each other about the joint(s), a magnetic field of said magnet body provides partial or more constraint at at least one of the degree(s) of freedom between the first body and the second body.

In another embodiment, a handheld surgical instrument retention assembly may include a handle body, a frame body, an articulation input joint, one or more retention bodies, and one or more retention holders. The frame body is coupled with the handle body. The articulation input joint is established between the handle body and the frame body. The articulation input joint provides a pitch rotational degree of freedom between the handle body and the frame body, and the articulation input joint provides a yaw rotational degree of freedom between the handle body and the frame body. Movements of the handle body with respect to the frame body by way of the articulation input joint and about the pitch and yaw rotational degrees of freedom serve to furnish corresponding movements at least one end effector of the accompanying handheld surgical instrument. The retention body(ies) is carried by the handle body or, alternatively, is carried by the frame body. The retention body(ies) is moveable, and can be moved, with respect to the handle body or with respect to the frame body at least between a retracted position and a deployed position. The retention holder(s) is located at the other of the handle body or the frame body, depending on which of the handle body or frame body the retention body is carried by. Upon movement of the retention body(ies) to its deployed position and engagement of the retention body(ies) with the retention holder(s), movements of the handle body with respect to the frame body by way of the articulation input joint and about the pitch and yaw rotational degrees of freedom are partially or more constrained and the corresponding movements of the end effector(s) are partially or more constrained.

Further scope of applicability of the present disclosure will become apparent from the detailed description given hereinafter. But it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The present disclosure will become more fully understood from the detailed description given below and the accompanying drawings, which are given by way of illustration only, and do not limit the present disclosure, and wherein:

FIG. 1 is a block diagram of an embodiment of a retention assembly;

FIG. 2 is a block diagram of another embodiment of a retention assembly;

FIG. 3 is a block diagram of another embodiment of a retention assembly;

FIG. 4 is a block diagram of another embodiment of a retention assembly;

FIG. 5 shows an embodiment of a handheld surgical instrument equipped with an embodiment of a handheld surgical instrument retention assembly;

FIG. 6 shows the handheld surgical instrument with the handheld surgical instrument retention assembly of FIG. 5;

FIG. 7A is a schematic diagram of an embodiment of a retention assembly of a positive engagement type, the retention assembly presented in an unlocked state (also called a first state or position);

FIG. 7B is a schematic diagram of the retention assembly of FIG. 7A presented in a partially engaged lock state (also called a second or intermediate state or position);

FIG. 7C is a schematic diagram of the retention assembly of FIG. 7A presented in a fully locked state (also called a third state or position);

FIG. 8A is an enlarged view of an embodiment of a handheld surgical instrument retention assembly presented in an unlocked state;

FIG. 8B is an enlarged view of the handheld surgical instrument retention assembly of FIG. 8A, the handheld surgical instrument retention assembly presented in a fully locked state;

FIG. 9 is an enlarged sectional view of interior components of an embodiment of a retention assembly;

FIG. 10 is a block diagram of another embodiment of a retention assembly;

FIG. 11 is a schematic diagram of an embodiment of a retention assembly of a non-positive engagement type, the retention assembly presented in a locked state;

FIG. 12 is a block diagram of another embodiment of a retention assembly;

FIG. 13A shows an embodiment of a handheld surgical instrument equipped with an embodiment of a handheld surgical instrument retention assembly;

FIG. 13B is an enlarged view of an embodiment of a retention holder located at a frame body of the handheld surgical instrument retention assembly;

FIG. 13C is a sectional view of another embodiment of a retention holder located at the frame body of the handheld surgical instrument retention assembly;

FIG. 14 is an enlarged view of an embodiment of an end effector assembly equipped with an embodiment of a shaft seal assembly;

FIG. 15 shows interior components of the end effector assembly and shaft seal assembly of FIG. 14;

FIG. 16 is a sectional view of another embodiment a shaft seal assembly equipped near an end effector assembly;

FIG. 17 is a cross-sectional view of the shaft seal assembly of FIG. 14; and

FIG. 18 is a cross-sectional view of the shaft seal assembly of FIG. 16.

### DETAILED DESCRIPTION

Multiple embodiments of retention assemblies and shaft seals and handheld surgical tools are depicted in the figures and detailed in this description. Definitions of certain technical terms used herein are presented prior to particular figure references in this description:

Body—A body is a discrete continuous component that can be used as structural components to form an assembly or sub-assembly. The displacement/motion state of a body can be completely defined with respect to a reference ground by six degrees of freedom (DoF). A body may be rigid or compliant. One or more discrete bodies may be connected via joints to form an assembly. These bodies, together as an assembly, may still be termed as a body. In certain scenarios, a body may be produced out of many discrete rigid bodies joined by rigid joints. The resulting body, alternatively, may also be a single/monolithic structure. In certain scenarios, a body may be compliant but still discrete and continuous.

Mechanism/Joint/Connector—In general, there may be a certain equivalence between the terms “mechanism” and “joint.” A “joint” may be alternatively referred to as a “connector” or a “constraint.” All of these can be viewed as allowing certain rigid body motion(s) along certain degree(s) of freedom between two bodies and constraining the remaining motions. A mechanism generally comprises multiple joints and bodies. Typically, a joint may be of simpler construction, while a mechanism may be more complex as it can comprise multiple joints. Generally, a joint refers to a mechanical connection that allows motions as opposed to a fixed joint (e.g., welded, bolted, screwed, or glued jointly). In the latter case, fixed joint, two bodies are fused with each other and are considered one and the same in the kinematic sense (because there is no relative motion allowed or there are no relative degrees of freedom between the two bodies). The term “fixed joint” may be used herein to refer to this kind of joint between two bodies. When reference to the term “joint” is made, it means a connection that allows at least some motions or degrees of freedom, e.g., a pin joint, a pivot joint, a universal joint, a ball and socket joint, etc.

Degree of Freedom (DoF)—As noted, a joint or mechanism allows certain rigid body motions between two bodies and constrains the remaining motions. “Degrees of freedom” is a technical term to capture or convey these allowed “motions.” In total, there are six independent motions and therefore six degrees of freedom possible between two rigid bodies when there is no joint between them: three translational DoFs and three rotational DoFs. A joint will allow anywhere between zero and six DoFs between the two bodies. For the case when the joint allows zero DoFs, this effectively becomes a “fixed joint,” as described above, where the two bodies are rigidly fused or connected to each other. In this case, from a kinematic sense, the two bodies are one and the same. For the case when the joint allows six DoFs, this effectively means that the joint does not constrain any motions between the two bodies. In other words, the motions of the two bodies are entirely independent of each other.

Degree of Constraint (DOC)—“Degree of constraint” refers to directions along which relative motion is constrained between two bodies. Since relative motion is constrained, these are directions along which motion and loads (i.e., forces or moments) can be transmitted from one body to the other body. Since the joint does not allow relative motion between the two bodies in the DoC direction, if one body moves in the DoC direction, it drives along with it the other body along that direction. In other words, motions are transmitted from one rigid body to another in the DoC directions. Consequently, loads are also transmitted from one rigid body to another in the DoC directions, which are sometimes also referred to as the load bearing directions or simply bearing directions. The term “retention” may also be used in the context of a DoC direction. For example, one body may be constrained or equivalently retained with respect to a second body along a certain DoC. This means that relative motion is not allowed between the two bodies in the DoC direction, or equivalently the direction of constraint, or equivalently the direction of retention. Retention of all six DoFs means the same thing as having six DoCs between two bodies.

Local Ground—In the context of an assembly of bodies connected by joints (e.g., a multi-body system, a mechanism), one or more bodies may be referred to as the “reference” or “ground” or “local ground.” The body referred to as the local ground is not necessarily an absolute ground (i.e., attached or bolted to the actual ground). Rather, the body that is selected as a local ground simply serves as a mechanical reference with respect to which the motions of all other bodies are described or investigated.

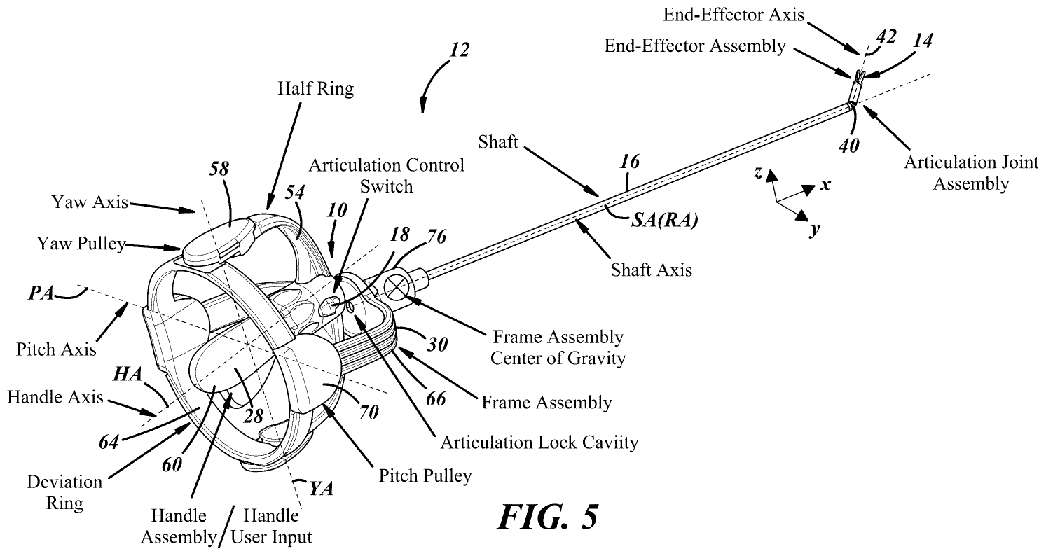

Axis and Direction-Axis refers to a specific line in space. A body may rotate with respect to (w.r.t.) another body about a certain axis. Alternatively, a body may translate w.r.t. another body in a certain direction. A direction is not defined by a particular axis, and rather is commonly defined by multiple parallel axes. Thus, x-axis is a specific axis defined in space, while X direction refers to the direction of the x-axis or any other axis that is parallel to the x-axis. Different but parallel axes can have the same X direction. Direction only has an orientation and not a location in space. In at least some embodiments, and with particular reference to FIG. 5, a coordinate system is presented with the x-axis coinciding with an axis of a shaft of the handheld surgical tool, the y-axis oriented relative thereto, and the z-axis oriented relative thereto. Here, the x-axis can also be referred to as a roll axis when the particular device rolls about the axis, the y-axis is established by a pitch axis and the z-axis is established by a yaw axis.

End Effector Assembly—When provided in an embodiment, the end-effector (EE) assembly may be referred to as the EE assembly, or simply the end-effector (EE). In some embodiments, the EE may exist at the distal end of the tool shaft. An EE assembly may be one of a variety of types that are useful in a surgical arena (e.g., needle holder, hook electrode, grasper, dissector, forceps, vessel sealer, clip applier, etc.). The EE may be further coupled to the device frame or device shaft via an output articulation joint.

User Interface—A user interface acts as an input interface that a user interacts with to produce certain output at a remotely located end of a machine or instrument or mechanism. User interface is generally an ergonomic feature on a body, which is part of an instrument, that is triggered by the user. The user interface may be referred to as the user input.

Retention Body, Feature, or Lock—Bodies may be attached via “retention features,” or “locking features” on the bodies that are retained together. Two or more bodies may also be retained via a third body, referred to as a “lock,” or a “lock body.” A lock has either an “ON” or an “OFF” status. These may be denoted as “1” or “0” respectively. A retention feature may be subject to contact pressure between two bodies that are retained together. For example, a detent on a first body and complementary mating feature on a second body provide retention between the two bodies, thus introducing one or more degrees of constraint between the bodies. These features are part of each respective body and are both examples of retention features. In other scenarios, there may be an external lock body, or third body that is retained to each body and effectively provides retainment between the first and second bodies. For example, a door latch (lock body) retains a door (first body) to a door frame (second body). In other words, the door is retained to the door frame via the door latch.

A retention feature or lock can be classified based on whether it provides positive engagement or non-positive engagement. A positive engagement lock refers to a mechanical retention feature between two bodies and is physically blocking the motion of one body with respect to the other body along the direction that is retained. A non-positive engagement lock does not have a feature that physically blocks relative motion but rather refers to a feature that uses friction (pressure between bodies), a magnetic field, or similar method to provide retention between the two bodies. A positive engagement locks may be designed such that it is backdrivable or non-backdrivable. A backdrivable lock can be undone, or unlocked, by pulling the two retained bodies apart from each other. A non-backdrivable lock cannot be undone by applying separation force on the two bodies that are retained. To undo a non-backdrivable lock, an external input is typically needed.

Transmission Member—A transmission member is a rigid or compliant body that transmits motions from an input body (produces input motion that is to be transmitted), to an output body (produces an output motion). The path that a transmission member takes impacts the efficiency of a transmission system.

Frame—When provided in an embodiment, the device frame, or simply frame, refers to a structural body or subassembly, which may have relative motion amongst components, that may be part of a tool apparatus or surgical instrument or tool. In certain tool apparatuses, it may be connected to a handle assembly and/or an elongated device shaft. Terms namely “device frame” and “frame” may be used interchangeably throughout the document.

Shaft—When provided in an embodiment, a device shaft, tool shaft, or simply shaft, is generally a rigid extension of the frame, at its proximal end, which is a slender and elongated member, commonly a cylinder. An output body, such as an end-effector assembly, may be constrained to the distal end of the shaft.

Unlimited Roll—An apparatus (i.e., instrument, device, tool, system, mechanism, etc.) that provides unrestricted roll, or rotation about a single axis, of a body within the apparatus. Bodies that have unlimited roll do not have a limitation in the amount of roll allowed. The roll may also be referred to as “infinite” or unrestrained, or full rotational capabilities.

Partial Constraint—Partial constraint is intended to embrace bodies that are retained to each other via positive engagement and non-positive engagement, as well as arrangements in which guided motion is effected between the bodies. For example, at least partial constraint encompasses: i) a pair of bodies that are fully constrained with respect to each other and hence exhibit six degrees of constraint (DOC) therebetween; ii) a pair of bodies retained by way of external input such as application of a magnetic field and/or retained by way of friction generated between the bodies; and iii) a pair of bodies in which motion of one body is guided along a motion path provided by the other body, and in which the bodies are otherwise constrained and retained from movement in other non-guided motions with respect to each other. Moreover, partial constraint refers to a constraint that can be interrupted by either force, pressure, or motion, typically from a user input. A partial constraint constrains bodies in certain scenarios and does not provide constraint in others. Further, a joint having a detent feature may also be referred to as a joint providing partial constraint between bodies.

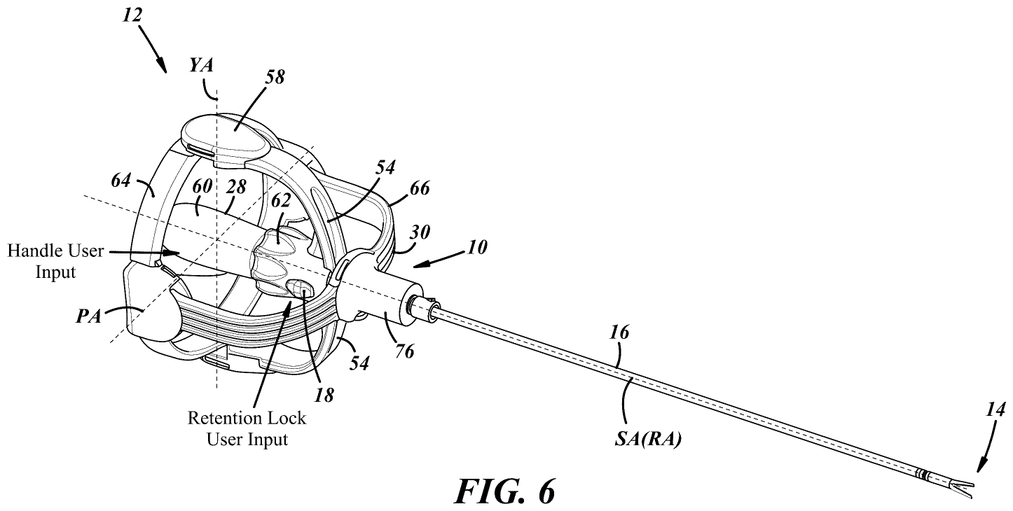

With reference now to the figures, embodiments of a retention assembly 10 are described and depicted herein. The retention assembly 10 can be equipped in a handheld surgical tool or instrument 12 or other medical device, as well as in non-medical devices, among other possible applications. When in the handheld surgical instrument 12, the retention assembly 10 is referred to as a handheld surgical instrument retention assembly 10 and can be employed for use in a minimally invasive surgical (MIS) procedure. In at least some embodiments presented below, and in the application of the handheld surgical instrument retention assembly 10 in use in an MIS procedure, the handheld surgical instrument retention assembly 10 can serve to constrain movement—and hence hold a position of—an end effector body 14 of the handheld surgical instrument 12 that otherwise is able to articulate in many positions. Maintaining the position of the end effector body 14, at least temporarily, can be useful at various times before and/or during and/or after an MIS procedure. For instance, the handheld surgical instrument retention assembly 10 can keep the end effector body 14 in a straight position relative to a shaft body 16 (e.g., see FIG. 6) for initial insertion of the end effector body 14 and shaft body 16 through a surgical trocar in a patient's body. The handheld surgical instrument retention assembly 10 thereby lessens or altogether relieves the effort, and sometimes burden, elsewise falling to a user of handheld surgical instrument 12 for performing the same function. In another example where such constraint and position maintenance can be useful, amid an MIS procedure the handheld surgical instrument retention assembly 10 can be employed to retract and/or separate and/or hold tissues or other bodily specimen away from a surgeon's field of view—here, the handheld surgical instrument retention assembly 10 can keep the end effector body 14 in an articulated position relative to the shaft body 16. Yet further, in certain embodiments this constraint and position maintenance may serve to facilitate the exchange of instruments and ends mounted for the end effector body 14. Still, other beneficial uses are possible in other embodiments and in other applications.

Furthermore, compared to certain past devices, according to some embodiments of the retention assembly 10 a control switch 18 exhibits more ready ergonomic accessibility and usability for a user of the larger device equipped with the retention assembly 10 such as the handheld surgical instrument 12; indeed, in embodiments the control switch 18 is actuatable via mere motion of a user's thumb or finger. Overall, a more effective and efficient retention assembly 10 is provided. Moreover, the retention assembly 10 can have various designs, constructions, and components in various embodiments depending upon, among other possible factors, the application in which the retention assembly 10 is employed in use and the desired functionalities to be effected via the retention assembly 10.

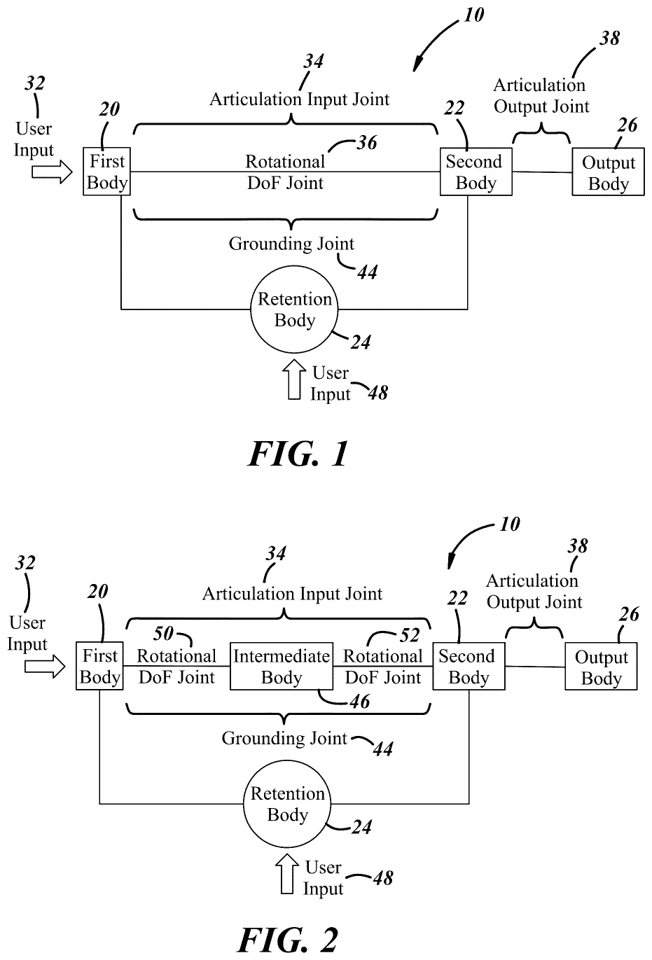

FIG. 1 presents an embodiment of the retention assembly 10 in block diagrammatic form. Here, the retention assembly 10 has a first body or assembly 20, a second body or assembly 22, a retention body or assembly 24, and an output body or assembly 26. The first body/assembly 20 and second body/assembly 22 can take various forms in various embodiments. The first body/assembly 20 and second body/assembly 22 can be coupled with each other. Depending on the embodiment and application of the retention assembly 10, the first body/assembly 20 can be a single, discrete body or an assembly of multiple discrete bodies joined together in various ways. Likewise, the second body/assembly 22 can be a single, discrete body or an assembly of multiple discrete bodies joined together in various ways. The first body/assembly 20 can include, or itself can constitute, an input body. In the example application of the handheld surgical instrument 12, for instance, and as depicted and described elsewhere, the first body/assembly 20 can be a handle body 28 and the second body/assembly 22 can be a frame body 30. The handle body 28 can constitute a user interface. User input 32 can be provided at the first body/assembly 20 by the user of the retention assembly 10. In the example application of the handheld surgical instrument 12, a surgeon or other user of the handheld surgical instrument 12 exerts the user input 32. The user input 32 can be a manipulation of the handle body 28 such as articulation input motions, rigid body motions, and/or roll input motions, among other possible user inputs. The articulation input motions can be pitch articulation input motions, yaw articulation input motions, or both pitch and yaw articulation input motions, per various embodiments. As depicted in FIG. 5, the rigid body motions can be along an x-axis or shaft axis SA, can be along a y-axis (e.g., right and left movements), and/or can be along a z-axis (e.g., up and down movements). The shaft axis SA is established by an elongated extent of the shaft body 16. The roll input motions are about the shaft axis SA.

Further, and with continued reference to FIG. 1, an articulation input joint 34 is established between the first body/assembly 20 and the second body/assembly 22. The articulation input joint 34 can take various forms in various embodiments. The articulation input joint 34 resides at a region residing between the first body/assembly 20 and the second body/assembly 22. The pitch and/or yaw articulation input motions are effected at the articulation input joint 34, and are independent with respect to each other. As described in more detail below in the example of the handheld surgical instrument 12, the articulation input joint 34 serves to capture the pitch and/or yaw articulation input motions between the first body/assembly 20 and the second body/assembly 22 for transference to the output body/assembly 26. The transference is effected via articulation transmission members (introduced below), per an embodiment. The degree of relative motion of the articulation input joint 34 relates proportionally to the magnitudes of the motions of the articulation transmission members. Further, when the motions of the articulation input joint 34 are constrained and restricted, the motions of the articulation transmission members are concomitantly constrained and restricted. The articulation input joint 34, per this embodiment, provides a rotational degree of freedom joint 36 between the first body/assembly 20 and second body/assembly 22. The rotational degree of freedom joint 36 can provide a pitch rotational degree of freedom between the first body/assembly 20 and the second body/assembly 22, can provide a yaw rotational degree of freedom between the first body/assembly 20 and the second body/assembly 22, or can provide both a pitch and a yaw rotational degree of freedom between the first body/assembly 20 and the second body/assembly 22.

Articulation input motions by the user input 32 between the first body/assembly 20 and second body/assembly 22 via the articulation input joint 34 and about the rotational degree of freedom joint 36 are transferred to the output body 26 by way of an articulation output joint 38. The articulation output joint 38 can take various forms in various embodiments. The articulation output joint 38 is established between the second body/assembly 22 and the output body 26 and extends therebetween. In the handheld surgical instrument 12 embodiment of FIG. 5, the articulation output joint 38 includes an articulation joint assembly 40. A multitude of multi-cluster articulation joints and components can be provided at the articulation output joint 38 and at the articulation joint assembly 40. The articulation output joint 38 permits an axis of the output body/assembly 26 to rotate with respect to the second body/assembly 22 (e.g., in the handheld surgical instrument 12, an end effector axis 42 is able to rotate with respect to the shaft body 16 and with respect to the shaft axis SA). Pitch and yaw rotations are permitted between the output body/assembly 26 and the second body/assembly 22 via the articulation output joint 38, while other degrees of freedom between the output body/assembly 26 and the second body/assembly 22 are restricted and constrained. Motions of the articulation output joint 38 are controlled by motions of the articulation transmission members. The articulation output joint 38 can be immobilized, or held in a rigid state, when motions of articulation transmission members are restricted and constrained simultaneously. Additional transmission members controlling motions of the output body/assembly 26 and the end effector body 14 in different ways can travel through the articulation output joint 38. Further, the articulation output joint 38 itself is generally incompressible when compressive loads are exerted between the output body/assembly 26 and the second body/assembly 22. The length and extent of the articulation output joint 38 is not altered and remains the same when pitch and yaw rotations occur. The articulation output joint 38 and articulation joint assembly 40 can be biased to a neutral position in which the axis of the output body/assembly 26 is in-line and coincident with the x-axis in the absence of input motions (e.g., in the handheld surgical instrument 12, the end effector axis 42 would be in-line and coincident with the shaft axis SA). Furthermore, per an embodiment, the articulation output joint 38 can be made-up of multiple joints provided in a series arrangement with a pitch DoF provided a one joint and a yaw DoF provided at another joint.

Furthermore, and with continued reference to FIG. 1, a grounding joint 44 resides between the first body/assembly 20 and the second body/assembly 22 according to this embodiment. In general, each body of the retention assembly 10 and of the handheld surgical instrument 12 exhibits an interface with adjacent bodies and can transfer motions via their degrees of constraint. When bodies are situated in a series arrangement, the total degrees of freedom between the initial body of the series and the final body of the series is equivalent to the addition of the unique degrees of freedom provided throughout the larger system of series of bodies. A grounding joint in this example is a joint that conveys rigid body motions from one body to another body. The grounding joint can translate one or more degrees of freedom from one body to another body. The grounding joint serving to convey motions is the same as transmitting motions from one body to another body. In FIG. 1, rigid body motions are conveyed from the first body/assembly 20 and to the second body/assembly 22 via rigid body motions conveyed through the grounding joint 44. Furthermore, per an embodiment, when the retention body/assembly 24 is deactivated and in an unlocked state, and/or the control switch 18 is OFF, the first body/assembly 20 can convey a number of degrees of freedom from the first body/assembly 20 to the second body/assembly 22: axial motion along the x-axis, y-axis position, z-axis position, and roll axis rotation. When the retention body/assembly 24 is activated and in a locked state, and/or the control switch 18 is ON, the first body/assembly 20 can convey an additional two degrees of freedom from the first body/assembly 20 to the second body/assembly 22: pitch rotation and yaw rotation. In other words, the grounding joint 44 is fully constrained via six DoC.

Referring still to FIG. 1, the retention body/assembly 24 can also be called a retention lock, an articulation lock, a switch, or simply a lock, according to various embodiments of the retention assembly 10. The retention body/assembly 24 can take various forms in various embodiments. In embodiments, the retention body/assembly 24 can be of the positive engagement lock type or of the non-positive engagement lock type. The retention body/assembly 24 has a location between the first body/assembly 20 and the second body/assembly 22 whereby, per an embodiment, upon its employment and actuation, the retention body/assembly 24 can effect a mechanical connection between the first body/assembly 20 and the second body/assembly 22. A constraint is effected between the first body/assembly 20 and the second body/assembly 22 via the retention body/assembly 24. When the mechanical connection is made, the retention body/assembly 24 can extend between the first body/assembly 20 and the second body/assembly 22. The retention body/assembly 24 can be located at and carried by the first body/assembly 20, or can be located at and carried by the second body/assembly 22. The retention body/assembly 24 can be controlled, or actuated, by way of the user interface and by way of the control switch 18. Further, in an embodiment, the retention body/assembly 24 can exhibit a bistable functionality. Here, the retention body/assembly 24 has two bistable positional states where the retention body/assembly 24 is in equilibrium. The two bistable positional states—e.g., first and second positions or states—can be referred to as ON and OFF states, activated and deactivated state, and/or locked and unlocked states. In this embodiment, the retention body/assembly 24 can be located at interstitial positions between the two bistable positional states only when external forces are present, such as when switching between the two bistable positional states. When such external forces are absent, the retention body/assembly 24 biases toward one of its two bistable positional states due to internal mechanism bias.

Further, the retention body/assembly 24 includes the control switch 18, per an embodiment, which can also be an articulation control switch that is user activated. In an embodiment, the retention body/assembly 24 can be an articulation lock feature that interacts with a complementary articulation lock receptacle. In the handheld surgical instrument 12 embodiment of FIG. 5, the retention body/assembly 24 can be brought to its ON state when a handle axis HA is aligned with and has a coincident arrangement with the shaft axis SA (such alignment is depicted in FIG. 6). The handle axis HA is established by an elongated extent of the handle body 28. When unaligned and not coincident, according to an embodiment, the retention body/assembly 24 is precluded and prevented from being brought to its ON state. Here, efforts to activate the retention body/assembly 24 when unaligned results in the control switch 18 returning to the OFF or deactivated state due to the bistable functionality, per that embodiment.

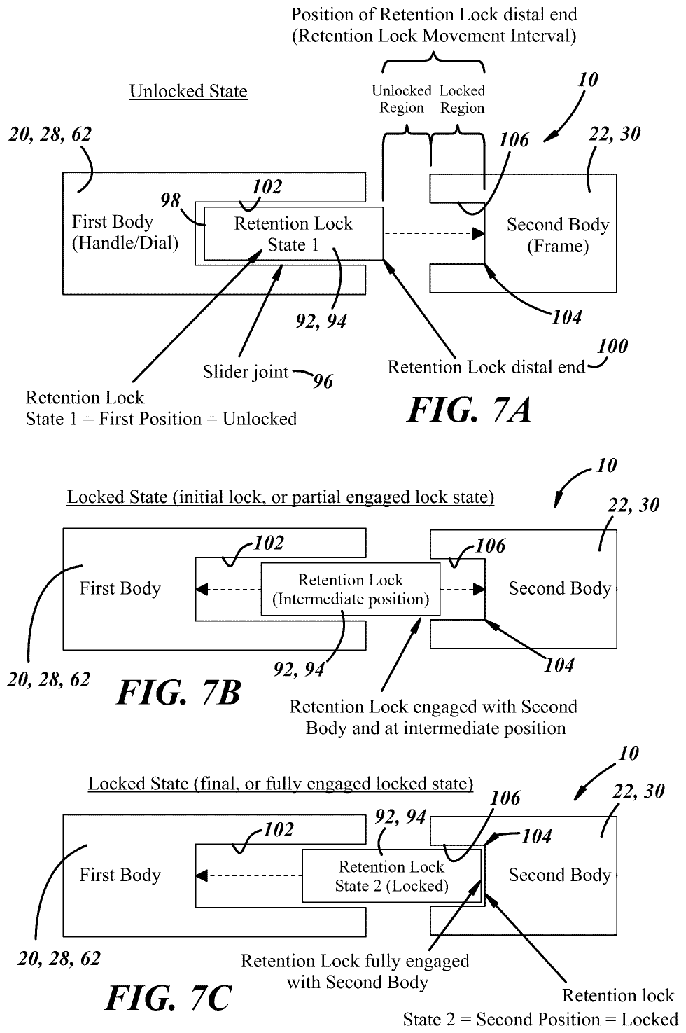

Furthermore, when the retention body/assembly 24 is in the ON and activated state, a feature such as the retention body/assembly 24 itself can physically and mechanically extend between the first body/assembly 20 and the second body/assembly 22, and can span a clearance and gap otherwise residing between the first body/assembly 20 and the second body/assembly 22. This is also referred to as a deployed position of the retention body/assembly 24, as shown in FIG. 7C, for example; an opposite, retracted position of the retention body/assembly 24 is shown in FIG. 7A, for example. The first body/assembly 20 and the second body/assembly 22 are thereby mechanically connected to each other. Although the mechanical connection bypasses an intermediate body or assembly 46 (FIG. 2) in its extension between the first body/assembly 20 and the second body/assembly 22, the mechanical connection works to immobilize the intermediate body/assembly 46. Further, the control switch 18 and retention body/assembly 24 can be connected together mechanically and/or electrically (e.g., button that electrically actuates the retention body/assembly), per various embodiments. When the mechanical connection is made between the first body/assembly 20 and the second body/assembly 22 via the retention body/assembly 24, the pitch rotational degree of freedom between the first body/assembly 20 and the second body/assembly 22, the yaw rotational degree of freedom between the first body/assembly 20 and the second body/assembly 22, or both the pitch and yaw rotational degrees of freedom between the first body/assembly 20 and the second body/assembly 22 can be constrained. In the embodiment of both the pitch and yaw rotational degrees of freedom between the first body/assembly 20 and the second body/assembly 22 being constrained, the articulation input joint 34 is thereby constrained. Such constraint by the retention body/assembly 24 also serves to constrain transmission member inputs (introduced below), thereby fully constraining movement of the output body/assembly 26 by means of fully constraining the articulation output joint 38. Further, when the retention body/assembly 24 is in the ON and activated state, rigid body motions of the output body/assembly 26 can be controlled by control of rigid body motions of the input body which, in the embodiment of the handheld surgical instrument 12, can be the handle body 28. When the retention body/assembly 24 is in the ON and activated state, all movements and motions of the output body/assembly 26 can be constrained. Moreover, in the handheld surgical instrument 12 embodiment of FIG. 5, when the retention body/assembly 24 is in the ON and activated state, the handle body 28 becomes fully constrained to the frame body 30 (i.e., six degrees of constraint are provided between the bodies). The end effector body 14 is hence also fully constrained and immobilized. Furthermore, in an embodiment, the control switch 18 need not necessarily be provided at the handle body 28, and rather could be equipped at a wireless receiver that receives user input from a remote location and, in turn, relays and electrically communicates a signal to the retention body/assembly 24 for activation or deactivation.

Still with reference to FIG. 1, user input 48 can be provided at the retention body/assembly 24 and to the control switch 18 by the user of the retention assembly 10. In the example application of the handheld surgical instrument 12, a surgeon or other user of the handheld surgical instrument 12 exerts the user input 48. As previously set forth, the user input 48 can be manipulation of the control switch 18 via the user's thumb or index finger. The control switch 18 can be toggled and moved back-and-forth, per an embodiment, to bring the retention body/assembly 24 between its ON and OFF states. The control switch 18 can be a button, switch, slider, knob, or the like. The control switch 18 serves to move the retention body/assembly 24 between its retracted and deployed positions. A slider joint, or some other joint or connection, can extend between the control switch 18 and the retention body/assembly 24. Further, in the embodiment of FIG. 5, the control switch 18 is located at, and carried by, the handle body 28 for ready user accessibility; in another embodiment the control switch 18 can be located at, and carried by, the frame body 30; still, other locations are possible. The control switch 18 can be positioned in the vicinity of a primary control user interface for ready and ergonomic user accessibility. When at the handle body 28, the control switch 18 is moved upon actuation with respect to the handle body 28. The control switch 18 can be conveniently actuated when the user has a grasp of the handle body 28. Moreover, at the handle body 28, the control switch could be accessible and actuatable by a user from two locations on the handle body 28 opposite relative to each other and one-hundred-and-eighty degrees (180°) apart. Amid actuation, the back-and-forth movement of the control switch 18, per this embodiment, can be in the X direction and along the x-axis or, in an embodiment in which it is located at the handle body 28, the back-and-forth movement can be along the handle axis HA. Actuation force needed for moving the control switch 18 can be optimized so that the control switch 18 is not inadvertently actuated, and so that it is not too difficult to actuated for certain users. The control switch 18 can be of any color and/or shape and/or design that serves to draw a user's attention and that can differentiate the control switch 18 from other surrounding components. Lastly, the control switch 18 can be located at a position whereby it does not impede or otherwise interfere with other intended functions of the retention assembly 10 and of the handheld surgical instrument 12.

The output body/assembly 26 can also be called an end effector assembly or simply an end effector. The output body/assembly 26 can take various forms in various embodiments. The output body/assembly 26 can be coupled to a distal and terminal end of the second body/assembly 22 by way of the articulation output joint 38. The output body/assembly 26 can itself constitute a distal and terminal end of the retention assembly 10. Position and orientation of the output body/assembly 26 relative to the second body/assembly 22 is determined by the position and orientation of the articulation output joint 38. Further, the position and orientation of the output body/assembly 26 with respect to the second body/assembly 22 can be fixed when the articulation output joint 38 is fixed. In various embodiments, the output body/assembly 26 can include various designs, constructions, and components that perform various functions. For example, the output body/assembly 26 can be jaws that open and close for grasping objects, or can be scissor blades that open and close for severing objects, among other possibilities. Furthermore, the output body/assembly 26 provides rigid termination and mounting points for pitch and yaw articulation transmission members and control cables (introduced below), according to an embodiment.

With reference now to FIG. 2, another embodiment of the retention assembly 10 is presented in block diagrammatic form. This embodiment shares many components described in connection with the embodiment of FIG. 1, the common descriptions of which may not be repeated here. In FIG. 2, the intermediate body/assembly 46 is situated between the first body/assembly 20 and the second body/assembly 22, and is coupled therebetween. The intermediate body/assembly 46 can take various forms in various embodiments. As shown by the depiction in FIG. 2, according to this embodiment, the retention body/assembly 24 can circumvent and physically and mechanically bypass the intermediate body/assembly 46 as the retention body/assembly 24 works directly and immediately between the first body/assembly 20 and the second body/assembly 22; in other words, the retention body/assembly 24 need not physically interact with the intermediate body/assembly 46 in order to perform its intended function of at least partial constraint between the first body/assembly 20 and the second body/assembly 22. Further, in this embodiment, a first rotational degree of freedom joint 50 is provided and extends between the first body/assembly 20 and the intermediate body/assembly 46, and a second rotational degree of freedom joint 52 is provided and extends between the second body/assembly 22 and the intermediate body/assembly 46. The first rotational degree of freedom joint 50 can provide a pitch rotational degree of freedom between the first body/assembly 20 and the intermediate body/assembly 46, or can provide a yaw rotational degree of freedom between the first body/assembly 20 and the intermediate body/assembly 46. Similarly, the second rotational degree of freedom joint 52 can provide a pitch rotational degree of freedom between the second body/assembly 22 and the intermediate body/assembly 46, or can provide a yaw rotational degree of freedom between the second body/assembly 22 and the intermediate body/assembly 46.

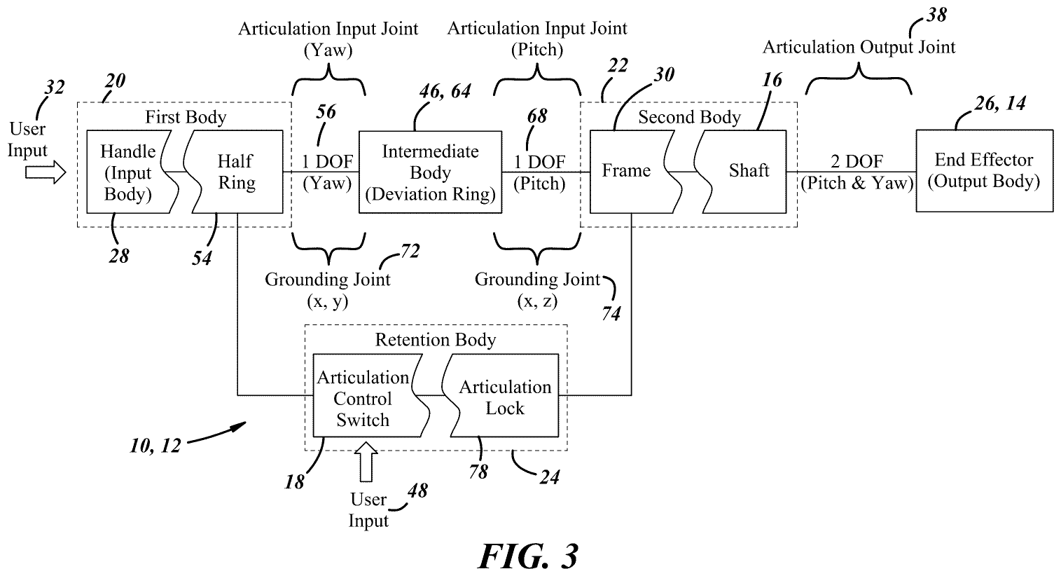

With reference now to FIG. 3, another embodiment of the retention assembly 10 and of the handheld surgical instrument 12 is presented in block diagrammatic form. This embodiment shares many components described in connection with the embodiment of FIGS. 1 and 2, the common descriptions of which may not be repeated here. In FIG. 3, the first body/assembly 20 is constituted by the handle body 28 and by a half ring body 54. The handle body 28 serves as a local ground for conveying rigid body motions to the larger retention assembly 10 and to the handheld surgical instrument 12. The half ring body 54 extends from the handle body 28, and has a fixed connection therewith. The half ring body 54 and the handle body 28 can be fully constrained with respect to each other; from a kinematics perspective, the half ring body 54 and handle body 28 can constitute a single body. The half ring body 54 is joined to the intermediate body/assembly 46 (here, a deviation ring body, introduced below), via a first joint which can be a set of pin joints. The first joint provides a yaw rotational degree of freedom 56 between the half ring body 54 and the deviation ring body.

Further, according to the embodiment of FIG. 5, a first or yaw pulley 58 is situated at one of the pin joints of the first joint and serves to capture yaw rotation of the half ring body 54 with respect to deviation ring body. The captured yaw rotation is transmitted to the output body/assembly 26 and to the end effector body 14 via the yaw articulation transmission members and control cables. The yaw articulation transmission members and control cables can be rigidly and fixedly mounted to the half ring body 54, per an embodiment. A yaw axis YA is established by the yaw rotational degree of freedom 56 and by an axis of rotation of the yaw pulley 58. The yaw axis YA exhibits an intersecting arrangement with the handle body 28. Furthermore, in the handheld surgical instrument 12 embodiment of FIG. 5, the handle body 28 includes a main body 60 and a dial body 62. The main body 60 can fit within a palm of a user's hand when the user is grasping the handle body 28 in use of the handheld surgical instrument 12. The dial body 62 serves as a user input for effecting unlimited roll functionality and capabilities at the end effector body 14. The dial body 62 can rotate about the handle axis HA with respect to the main body 60. The roll functionality of the end effector body 14 is with respect to, and about, the shaft axis SA. A roll axis RA can be established by the shaft axis SA.

With continued reference to FIG. 3, in this embodiment the intermediate body/assembly 46 is constituted by a full ring or deviation ring body 64. The pitch articulation transmission members and control cables can be rigidly and fixedly mounted to the deviation ring body 64, per an embodiment. The deviation ring body 64 furnishes an incompressible path for routing of the yaw articulation transmission members and control cables from the first body/assembly 20 and to the second body/assembly 22. Furthermore, in the handheld surgical instrument 12 embodiment of FIG. 5, the deviation ring body 64 is joined to a frame segment body 66 of the frame body 30 via a second joint which can be a set of pin joints. The second joint provides a pitch rotational degree of freedom 68 between the frame segment body 66 (and the frame body 30) and the deviation ring body 64. The frame segment body 66 extends from the frame body 30, and has a fixed connection therewith. The frame segment body 66 and the frame body 30 can be fully constrained with respect to each other; from a kinematics perspective, the frame segment body 66 and frame body 30 can constitute a single body. Further, according to the embodiment of FIG. 5, a second or pitch pulley 70 is situated at one of the pin joints of the second joint and serves to capture pitch rotation of the frame segment body 66 with respect to deviation ring body 64. The captured pitch rotation is transmitted to the output body/assembly 26 and to the end effector body 14 via the pitch articulation transmission members and control cables. A pitch axis PA is established by the pitch rotational degree of freedom 68 and by an axis of rotation of the pitch pulley 70. The pitch axis PA exhibits an intersecting arrangement with the handle body 28. Further, with reference again to FIG. 3, a first grounding joint 72 resides between the half ring body 54 and the deviation ring body 64, according to this embodiment, and a second grounding joint 74 resides between the frame segment body 66 and the deviation ring body 64.

In FIG. 3, the second body/assembly 22 is constituted by the frame body 30 and the shaft body 16. The shaft body 16 can be rigidly and fixedly mounted to the frame body 30 by way of a shaft mount body 76 (FIG. 5). The shaft body 16 itself can be rigid or compliant in bending. The shaft body 16 furnishes an incompressible path for routing of the yaw and pitch articulation transmission members and control cables, as well as a conduit therefor, to the articulation output joint 38. Further, in FIG. 3, the retention body/assembly 24 is constituted by the control switch 18 (denoted articulation control switch in the block diagram) and an articulation lock 78. The control switch 18 and the articulation lock 78 can be coupled together. The articulation lock 78 can be the retention body/assembly 24 itself.

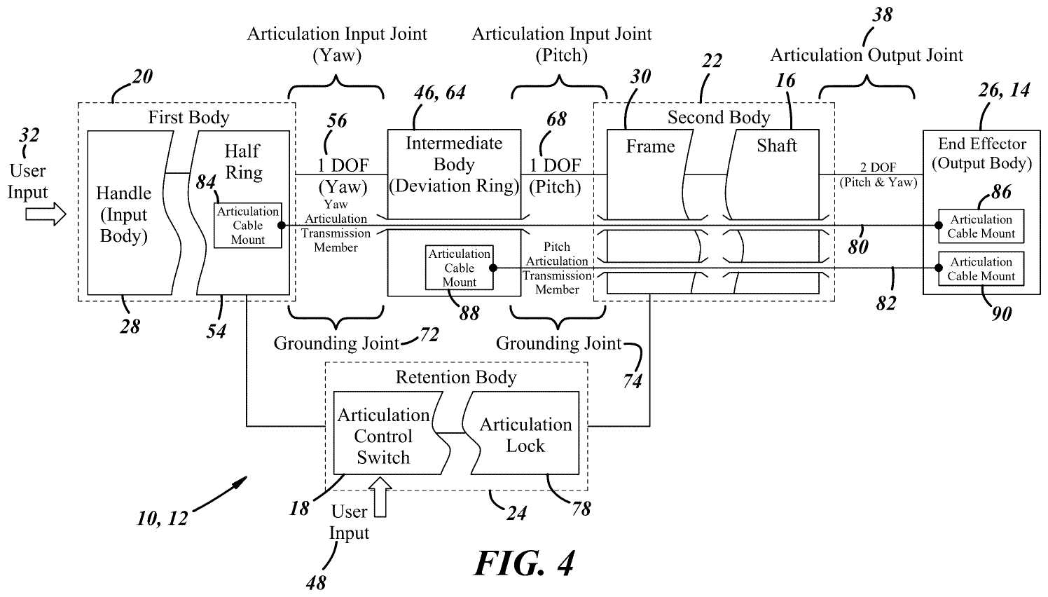

With reference now to FIG. 4, another embodiment of the retention assembly 10 and of the handheld surgical instrument 12 is presented in block diagrammatic form. This embodiment shares many components described in connection with the embodiment of FIGS. 1, 2, and 3, the common descriptions of which may not be repeated here. In FIG. 4, a yaw articulation transmission member and control cable 80 and a pitch articulation transmission member and control cable 82 are depicted. In general, in the block diagram of FIG. 4, transmission cable routing features are shown residing through the intermediate body/assembly 46 and deviation ring body 64, and through the second body/assembly 22 and frame body 30 and shaft body 16. The transmission cable routing features can provide cable routing surfaces while not constraining transmission member axial motion. The impact to transmission member motion can be limited to contact friction between the transmission member and the particular body. The yaw and pitch articulation transmission members 80, 82 can also be called transmission cables, control cables, elongate transmission members, articulation cables, or simply cables. In various embodiments, the yaw and pitch articulation transmission members 80, 82 can be stainless-steel braided wire ropes that act as a two-force tension member, may not support compressive loads, and may not transmit torque. The transmission members 80, 82, individually, can only transmit one energy input from an input to an output body. The transmission members transmit energy vis translational motion. Further, the yaw and pitch articulation transmission members 80, 82 can be routed via routing features that are integral to the bodies through which they are routed. The transmission members 80, 82 can slide or roll over routing feature such as channels or pulleys. Routing features generally do not significantly constrain translational motion of the yaw and pitch articulation transmission members 80, 82 through mechanisms such as surface friction. Sliding joints between the transmission members 80, 82 and the routing features can be akin to a cylindrical pair style of kinematic constraints whereby two axes of two bodies are aligned (i.e., four DoC).

With continued reference to FIG. 4, in this embodiment the yaw and pitch articulation transmission members 80, 82 terminate at fixed joints between their terminal extents. A pair of yaw articulation transmission members 80 can be provided, and a pair of pitch articulation transmission members 82 can be provided. Further, in the handheld surgical instrument 12 embodiment of FIG. 5, the yaw articulation transmission members 80 are coupled to the yaw pulley 58 at the half ring body 54 and provide input motions. A first articulation cable mount 84 (FIG. 4) of the yaw articulation transmission members 80 is located at the first body/assembly 20 and at the half ring body 54. The yaw articulation transmission members 80 are routed through channels residing in the intermediate body/assembly 46 and the deviation ring body 64, through channels residing in the second body/assembly 22 and the frame body 30, though the shaft body 16, through the articulation output joint 38, and to the output body/assembly 26 and to the end effector body 14. A second articulation cable mount 86 of the yaw articulation transmission members 80 is located at the output body/assembly 26 and the end effector body 14.

Similarly, the pitch articulation transmission members 82 are coupled to the pitch pulley 70 at the deviation ring body 64. A first articulation cable mount 88 (FIG. 4) of the pitch articulation transmission members 82 is located at the intermediate body/assembly 46 and the deviation ring body 64. The pitch articulation transmission members 82 are routed through channels residing in the second body/assembly 22 and the frame body 30, though the shaft body 16, through the articulation output joint 38, and to the output body/assembly 26 and to the end effector body 14. A second articulation cable mount 90 of the pitch articulation transmission members 82 is located at the output body/assembly 26 and the end effector body 14. Yaw and pitch input motions incite corresponding transmission member motions and cause corresponding end effector output motions. The magnitudes and motions of the yaw and pitch articulation transmission members 80, 82 at the articulation input joint 34 are generally equivalent to the magnitudes and motions of the yaw and pitch articulation transmission members 80, 82 at the articulation output joint 38. Furthermore, in general, an opposing pair of transmission members (both tension members) may constrain one DoF of an output body (i.e., one transmission member constrains positive rotational motion and an opposing transmission member constrains negative rotational motion). To fully constrain the motion of a pitch or yaw DoF at the articulation output joint 38, the motions of a pair of transmission members can be restricted.

Moreover, example handle bodies, frame bodies, and end effector bodies are depicted and described in U.S. Pat. No. 11,950,966 issued on Apr. 9, 2024 and owned by present applicant FlexDex, Inc., the contents of which are hereby incorporated by reference in their entirety. Further examples of handle bodies, frame bodies, and end effector bodies are depicted and described in U.S. Patent Application Publication No. 2023/0040475 published on Feb. 9, 2023 and owned by present applicant FlexDex, Inc., the contents of which are hereby incorporated by reference in their entirety.

With reference to the handheld surgical instrument 12 embodiment of FIG. 5, each body of the retention assembly 10 and of the handheld surgical instrument 12 can have a unique and independent center of gravity that can influence the motions of that particular body. For example, the center of gravity of the first body/assembly 20 is located within the handle body 28. And since the handle body 28 is the user input and is grasped by a user's hand, according to this embodiment, the center of gravity resides within the user's hand. The center of gravity of the second body/assembly 22, on the other hand, is located along the shaft axis SA and is distally located with respect to the first body/assembly 20. Due to the pitch and yaw rotational degrees of freedom between the first and second body/assembly 20, 22, the center of gravity of the frame body 30 causes the body to drop or point downward with respect to the handle body 28 when external influences are absent. In an embodiment, the degree that the frame body 30 is able to drop is determined by integral rotational travel stops at each of the accompanying rigid body grounding joints. Furthermore, the center of gravity for the handle body 28 and the frame body 30 are separate and independent with respect to each other. Without external forces, the motions of the handle body 28 and the frame body 30 are independent and influenced by the location of their respective centers of gravity.

Referring now to FIGS. 7A-7C, an embodiment of a positive engagement lock type of the retention assembly 10, and as implemented in the handheld surgical instrument 12, is shown schematically amid various states and positions. In this embodiment, the retention body/assembly 24 is in the form of a lock body 92 and a pin body 94. Here, the lock body 92 and pin body 94 are located at, and carried by, the first body/assembly 20 and the handle body 28. More specifically, the lock body 92 and pin body 94 are located at, and carried by, the dial body 62. Still, in other embodiments, the lock body 92 and pin body 94 could be located at, and carried by, the second body/assembly 22 and the frame body 30. The lock body 92 and pin body 94 exhibit a single degree of freedom with respect to the first body/assembly 20, handle body 28, and dial body 62. The single DoF according to this embodiment is a translational DoF. Accordingly, the lock body 92 and pin body 94 are translationally moveable with respect to the first body/assembly 20, handle body 28, and dial body 62. The lock body 92 and pin body 94 can move forward and rearward, and fore and aft, according to this embodiment. To facilitate such translational moveability therebetween, a slider joint 96 can be situated between the lock body 92 and pin body 94, and the first body/assembly 20, handle body 28, and dial body 62; still, other designs, constructions, and components for facilitation of movement between the bodies is possible in other embodiments. The lock body 92 and pin body 94 have an elongated unitary extension in the embodiment of FIGS. 7A-7C, and span from a proximal end 98 to a distal and terminal end 100. The lock body 92 and pin body 94 are seated and carried within a receptacle 102 residing within the first body/assembly 20, handle body 28, and dial body 62. The lock body 92 and pin body 94 move within the receptacle 102. The lock body 92 and pin body 94 can have a generally cylindrical shape, or another shape.

Furthermore, in this embodiment, the retention assembly 10 includes a retention holder 104. Here, the retention holder 104 resides in the second body/assembly 22 and the frame body 30; still, in the embodiment in which the lock body 92 and pin body 94 are located at the second body/assembly 22 and frame body 30, the retention holder 104 could reside in the first body/assembly 20 and handle body 28. The retention holder 104 can be situated in general confrontation and opposition with the lock body 92 and pin body 94, and particularly when the handle axis HA and shaft axis SA exhibit a coincident and aligned arrangement with respect to each other in the embodiment of the handheld surgical instrument 12. Such coincident and aligned arrangement is demonstrated in FIGS. 6, 7A-7C, 8A, and 8B. The retention holder 104 is in the form of a cavity 106, per this embodiment (cavity is also shown in FIG. 5). The cavity 106 receives insertion of the lock body 92 and pin body 94 when engagement is carried out between the components. The cavity 106 has an open end that initially receives the lock body 92 and pin body 94, and a closed end situated opposite its open end. The cavity 106 can have a shape and size that is generally complementary to that of the lock body 92 and pin body 94 in order to furnish a close-fit therebetween upon their engagement. In an alternative embodiment, the lock body could have extensions spanning from first body/assembly 20 and engaging with external/outside surfaces of the second body/assembly 22.

In FIG. 7A, the retention assembly 10 is depicted in an unlocked state, also called a first state or a first position or positional state of the retention assembly 10. The lock body 92 and pin body 94 are in a fully retracted position. Insertion and reception between the lock body 92 and pin body 94 and the cavity 106 is lacking. The components are wholly disengaged. Constraint between the first body/assembly 20, handle body 28, and dial body 62 and the second body/assembly 22 and frame body 30 due to the retention assembly 10 is absent. A clearance and gap—otherwise bridged by the lock body 92 and pin body 94 when fully deployed and engaged with the cavity 106—resides between the first body/assembly 20, handle body 28, and dial body 62 and the second body/assembly 22 and frame body 30. In FIG. 7B, the retention assembly 10 is depicted in a partially engaged lock state, also called a second state or a second position or positional state of the retention assembly 10. The lock body 92 and pin body 94 are in a partially deployed position. Insertion and reception between the lock body 92 and pin body 94 and the cavity 106 is effected, with a distal end portion of the lock body 92 and pin body 94 received in the cavity 106. The components are partially engaged. Constraint between the first body/assembly 20, handle body 28, and dial body 62 and the second body/assembly 22 and frame body 30 due to the retention assembly 10 is effected. The constraint between the components is full constraint per this embodiment. When effected—and upon constraint between the first body/assembly 20, handle body 28, and dial body 62 and the second body/assembly 22 and frame body 30—in the embodiment of the handheld surgical instrument 12, the half ring body 54 and deviation ring body 64 are also constrained with respect to the second body/assembly 22 and frame body 30. Moreover, as a consequence, the yaw and pitch articulation transmission members 80, 82 are restricted from movement, and hence articulation movements at the output body/assembly 26 and end effector body 14 via the articulation output joint 38 are restricted and constrained. The articulation movements of the output body/assembly 26 and end effector body 14 are effectively rendered immobile.

In FIG. 7C, the retention assembly 10 is depicted in a fully engaged lock state, also called a third state or a third position or positional state of the retention assembly 10. The lock body 92 and pin body 94 are in a fully deployed position. Insertion and reception between the lock body 92 and pin body 94 and the cavity 106 is fully effected. The components are fully engaged. Constraint between the first body/assembly 20, handle body 28, and dial body 62 and the second body/assembly 22 and frame body 30 due to the retention assembly 10 is effected. The constraint between the components is full constraint per this embodiment. As before, when effected—and upon constraint between the first body/assembly 20, handle body 28, and dial body 62 and the second body/assembly 22 and frame body 30—in the embodiment of the handheld surgical instrument 12, the half ring body 54 and deviation ring body 64 are also constrained with respect to the second body/assembly 22 and frame body 30. Moreover, as a consequence, the yaw and pitch articulation transmission members 80, 82 are restricted from movement, and hence articulation movements at the output body/assembly 26 and end effector body 14 via the articulation output joint 38 are restricted and constrained. The articulation movements of the output body/assembly 26 and end effector body 14 are effectively rendered immobile. Lastly, between the first state or position or positional state of the retention assembly 10 and the third state or position or positional state, the lock body 92 and pin body 94 can have a multitude of positional states.

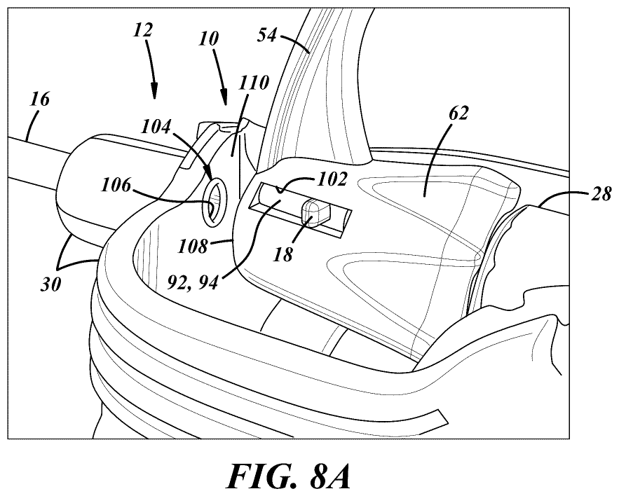

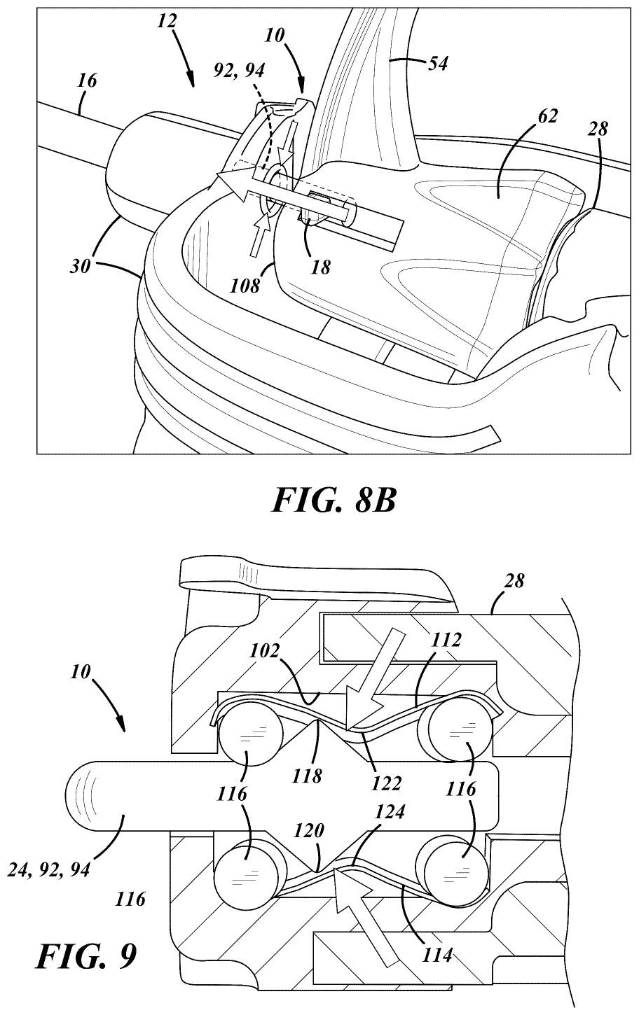

With reference now to FIGS. 8A and 8B, the embodiment of the positive engagement lock type of the retention assembly 10 is shown more specifically implemented in the handheld surgical instrument 12. Again here, the retention body/assembly 24 is in the form of the lock body 92 and pin body 94, and are located at, and carried by, the first body/assembly 20 and handle body 28 and dial body 62. The lock body 92 and pin body 94 exhibit a single translational DoF with respect to the first body/assembly 20 and handle body 28 and dial body 62. The single translational DoF in this embodiment is aligned with the handle axis HA, and hence the lock body 92 and pin body 94 are deployed and retracted back-and-forth in-line with the handle axis HA. When in the fully retracted position as shown in FIG. 8A, the lock body 92 and pin body 94 are fully seated within the receptacle 102, and the distal end 100 of the lock body 92 and pin body 94 can be positioned flush or withdrawn back from an end surface 108 of the dial body 62. Further, and again here, the retention holder 104 is in the form of the cavity 106. The retention holder 104 is located at an inward or rearside surface 110 of the frame body 30, and the cavity 106 resides therein. When in the fully deployed position as shown in FIG. 8B, the lock body 92 and pin body 94 are fully extended and suspended and projected beyond the end surface 108 of the dial body 62. The lock body 92 and pin body 94 are fully inserted into the cavity 106, as demonstrated by the broken line depiction in FIG. 8B. Furthermore, the embodiment of FIGS. 8A and 8B provides a single cavity 106 that is set in alignment with the shaft axis SA and that, when engaged by the lock body 92 and pin body 94, furnishes a constraint in which the handle axis HA is retained in a coincident arrangement with the shaft axis SA and the end effector axis 42 is hence also in alignment with the shaft axis SA. Still, in alternative embodiments, there could be a multitude of discrete cavities at the frame body that are set at various radial positions relative to the single cavity of FIGS. 8A and 8B and that are misaligned and offset with respect to the shaft axis SA. Here, when one of these radially-positioned cavities are engaged by the lock body and pin body, a constraint in which the handle axis HA is retained in a misalignment arrangement with the shaft axis SA is furnished. The end effector axis would hence also be in misalignment with the shaft axis SA, and the end effector would be retained in an articulated position relative to the shaft axis SA.

With reference now to FIG. 9, an embodiment of the retention assembly 10 is presented that exhibits bistable functionality. The bistable functionality can be carried out in various ways and can take various forms in various embodiments. In the embodiment of FIG. 9, as an example, the retention body/assembly 24 is in the form of the lock body 92 and pin body 94. The lock body 92 and pin body 94 are located at, and carried by, the first body/assembly 20 and handle body 28 and dial body 62, but could be located at the second body/assembly 22 and frame body 30. A pair of spring clips are situated within the receptacle 102 in this embodiment: a first spring clip 112 and a second spring clip 114. The spring clips 112, 114 constitute a detent mechanism. The spring clips 112, 114 can be composed of a metal material and can exhibit a degree of compliancy. Pegs 116 are provided at the receptacle 102 and at ends of the first and second spring clips 112, 114 in order to keep the spring clips in place. The first and second spring clips 112, 114 interact and engage the lock body 92 and pin body 94 to effect the bistable functionality. In FIG. 9, the lock body 92 and pin body 94 have a first protuberance 118 and a second protuberance 120 that are engaged and abutted by a first bend 122 and a second bend 124 of the first and second spring clips 112, 114. The first and second protuberances 118, 120 are outwardly projecting portions of the lock body 92 and pin body 94 relative to main portions thereof. In this embodiment, the first and second protuberances 118, 120 have forward and rearward angled and generally planar surfaces that make surface-to-surface engagement with opposing and confronting surfaces of the first and second spring clips 112, 114 and their first and second bends 122, 124. The engagement and abutment serves to yieldably bias the lock body 92 and pin body 94 between a first or fully deployed positional state at a forward side of the first and second bends 122, 124 (depicted in FIG. 9), and a second or fully retracted positional state at a rearward side of the first and second bends 122, 124 (not specifically shown). Still, other bistable positional states, including more in quantity, are possible in other embodiments. Furthermore, in an alternative to this embodiment, the retention body/assembly could be yieldably biased toward its second or fully retracted positional state, such as via a detent mechanism like one or more spring clips; here, the user would have to physically urge the retention body/assembly to its first or fully deployed positional state.

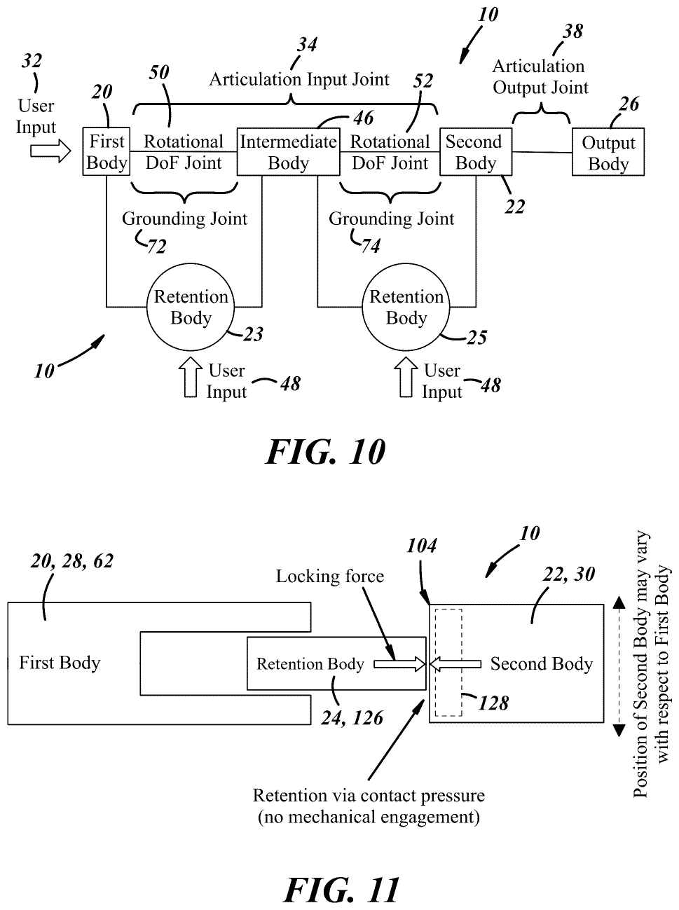

With reference now to FIG. 10, another embodiment of the retention assembly 10 and of the handheld surgical instrument 12 is presented in block diagrammatic form. This embodiment shares many components described in connection with the embodiment of FIGS. 1-4, the common descriptions of which may not be repeated here. In FIG. 10, a first retention body/assembly 23 and a second retention body/assembly 25 are provided. Correspondingly, a first retention holder and a second retention holder would be provided. The first and second retention body/assembly 23, 25 are independent with respect to each other, and can be individually activated and deactivated. As previously set forth, the first and second retention body/assembly 23, 25 can take various forms in various embodiments, including the various embodiments described elsewhere in this description. In embodiments, for instance, the first and second retention body/assembly 23, 25 can be of the positive engagement lock type or of the non-positive engagement lock type. Moreover, the type and form of the first retention body/assembly 23 need not be the same type and form of that of the second retention body/assembly 25. In FIG. 10, the first retention body/assembly 23 has a first location between the first body/assembly 20 and the intermediate body/assembly 46, and the second retention body/assembly 25 has a second location between the intermediate body/assembly 46 and the second body/assembly 22. In the embodiment of the handheld surgical instrument 12, the first retention body/assembly 23 is situated between the handle body 28/half ring body 54 and the deviation ring body 64, and the second retention body/assembly 25 is situated between the deviation ring body 64 and the frame body 30. In this embodiment, a first constraint is effected between the first body/assembly 20 and the intermediate body/assembly 46 via the first retention body/assembly 23, and a second constraint is effected between the intermediate body/assembly 46 and the second body/assembly 22. The first and second constraints are independent with respect to each other. Furthermore, in the embodiment of the handheld surgical instrument 12, the first constraint is effected between the handle body 28 and half ring body 54 and the deviation ring body 64, and the second constraint is effected between the deviation ring body 64 and the frame body 30. Yet further, in alternatives to this embodiment, only one of the first or second retention body/assembly 23, 25 need be provided at its respective first or second location.

With reference now to FIG. 11, an embodiment of a non-positive engagement lock type of the retention assembly 10 is shown that can be implemented in the handheld surgical instrument 12. In this embodiment, the non-positive engagement lock type can employ the use of magnetism or can employ the use of pressurization or forceful engagement. In the use of magnetism, the retention assembly 10 can include the retention body/assembly 24 in the form of a pin body 126, and can include the retention holder 104 in the form of one or more magnet bodies 128. The constraint effected between the first body/assembly 20 and second body/assembly 22 can have varying degrees of magnitude, depending on the locations of the pin body 126 and magnet body(ies) 128 relative to each other and depending on the distances between them. In FIG. 11, the pin body 126 is located at, and carried by, the first body/assembly 20 and handle body 28 and dial body 62; still, in other embodiments the pin body could be located at the second body/assembly and frame body. Similar to previous embodiments, the pin body 126 exhibits a single translational DoF with respect to the first body/assembly 20 and handle body 28 and dial body 62. For attraction to the magnet body(ies) 128, the pin body 126 can be composed of a metallic material and/or a ferromagnetic material, or itself could be a magnet. Further, in this embodiment the magnet body 128 is located at, and carried by, the second body/assembly 22 and frame body 30; still, in other embodiments the magnet body could be located at the first body/assembly and handle body and dial body, or yet further a first and second discrete magnet bodies could be situated at each location. The magnet body 128 can be a permanent magnet. The magnet body 128 can produce a magnetic field in the direction of the first body/assembly 20 and handle body 28 and dial body 62, and toward the pin body 126. When the pin body 126 begins its translational movement toward the magnet body 128, the magnetic field draws and pulls the pin body 126 toward the magnet body 128. The pin body 126 is shown in the fully deployed position in FIG. 11. Furthermore, in an embodiment, the magnet body 128 can be enlarged to span an increased radial extension and area at the rearside surface 110 (or other surface) in order to furnish a constraint in which the handle axis HA is retained in a misalignment arrangement with the shaft axis SA. In a yet further embodiment, the magnet body could be in the form of a spring-loaded detent pin, and a multitude of corresponding detent pin pockets could oppose the magnet body in this embodiment for producing a magnetic field and engagement therebetween. Moreover, in an alternative embodiment that employs the use of pressurization or forceful engagement, a friction-generating material could be situated at a distal end of the retention body/assembly 24 and/or could be situated at a confronting surface of the retention holder 104. When contact pressure or other forceful engagement occurs therebetween, a constraint is effected between the first body/assembly 20 and second body/assembly 22. Likewise here, a constraint in which the handle axis HA is retained in a misalignment arrangement with the shaft axis SA can be furnished.

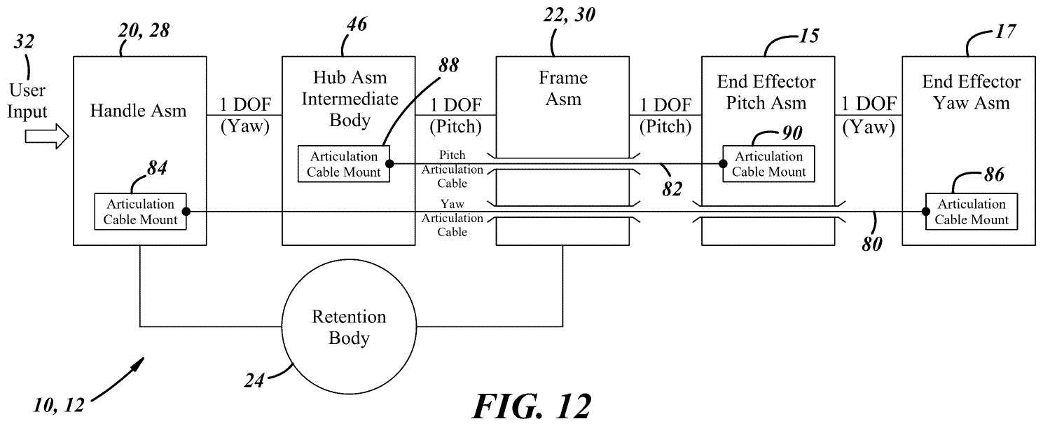

With reference now to FIG. 12, another embodiment of the retention assembly 10 and of the handheld surgical instrument 12 is presented in block diagrammatic form. This embodiment shares many components described in connection with previous embodiments, the common descriptions of which may not be repeated here. In FIG. 12, the output body/assembly and end effector include a first end effector assembly 15 and a second end effector assembly 17. The first and second end effector assemblies 15, 17 are not necessarily discrete end effectors, but rather can be assemblies of a single end effector body. In an embodiment, the first end effector assembly 15 is an end effector pitch assembly, and the second end effector assembly 17 is an end effector yaw assembly. The end effector pitch assembly and end effector yaw assembly exhibit a series arrangement with respect to each other, according to this embodiment, and can have an end effector tool secured to a distal end and portion of the second end effector assembly 17. In a more specific embodiment, the end effector pitch assembly is a pitch pulley of an end effector, and the end effector yaw assembly is a yaw pulley of an end effector. Further, the retention body/assembly 24 has a location between the first body/assembly 20 and the second body/assembly 22. In the embodiment of the handheld surgical instrument 12, the retention body/assembly 24 is situated between the handle body 28 and the frame body 30. Here, as before, a constraint is effected between the first body/assembly 20 and second body/assembly 22 via the retention body/assembly 24. In the embodiment of the handheld surgical instrument 12, the constraint is effected between the handle body 28 and the frame body 30, and serves to constrain movement, and hence immobilize, both the first and second end effector assemblies 15, 17. In this embodiment, the handle body 28 and frame body 30 can exhibit various configurations relative to each other and need not have the accompanying handle axis and shaft axis aligned and coincident with each other.

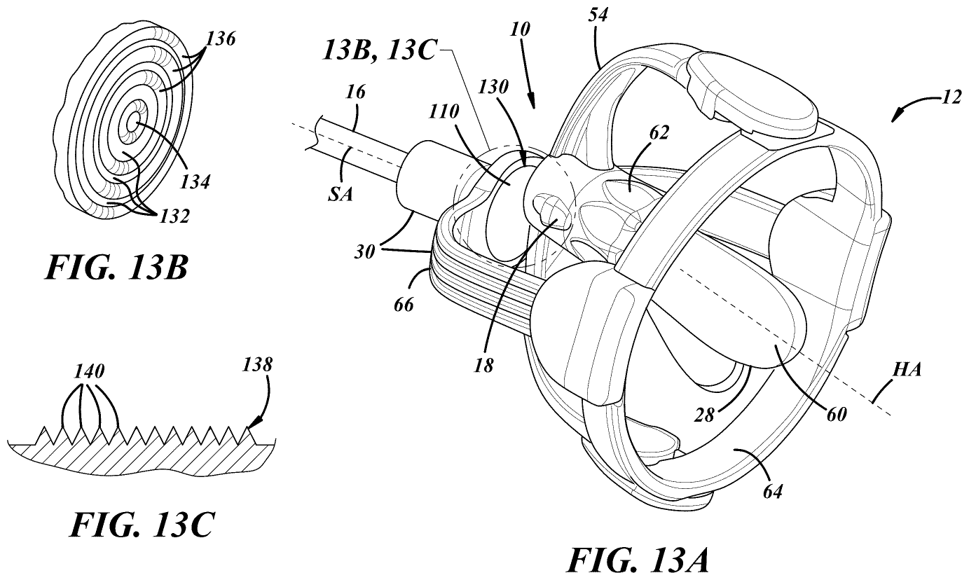

With reference now to FIGS. 13A-13C, additional embodiments of the retention assembly 10 and of the handheld surgical instrument 12 are presented. This embodiment shares many components described in connection with previous embodiments, the common descriptions of which may not be repeated here. In the embodiment of FIGS. 13A-13C, a constraint in which the handle axis HA is retained in a coincident arrangement with the shaft axis SA and the end effector body 14 is correspondingly retained is furnished, as well as a constraint in which the handle axis HA is retained in one of a multitude of misalignment arrangements with the shaft axis SA is furnished. With particular reference to FIGS. 13A and 13B, in this embodiment the retention body/assembly 24 is in the form of the lock body 92 and pin body 94 that is located at, and carried by, the first body/assembly 20 and handle body 28 and dial body 62, as previously described. The retention holder 104 includes a retention domain 130 located at the second body/assembly 22 and frame body 30. Still, in other embodiments, the locations of the retention body/assembly 24 and retention holder 104 can be interchanged with each other.

In FIG. 13A, the retention domain 130 can be constituted by a somewhat expansive portion and area at the rearside surface 110 (or other surface) of the second body/assembly 22 and frame body 30. The retention domain 130 resides in general confrontation with the retention body/assembly 24 for engagement therebetween. With particular reference now to FIG. 13B, according to this embodiment, the retention holder 104 and retention domain 130 includes one or more grooves 132 and a cavity 134. The retention body/assembly 24 can have a distal end complementarily shaped and sized for reception and retention within the grooves and cavity 134. Here, there are three grooves 132 that are circular in extent and ring-shaped; still, other quantities of grooves and/or other shapes and/or other arrangements are possible in other embodiments (e.g., radially-extending grooves, wide grooves, elliptical grooves, etc.). The grooves 132 are concentric with respect to the centrally-located cavity 134. The grooves 132 are concentric with the shaft axis SA, and the cavity 134 is aligned and concentric with the shaft axis SA. The grooves 132 are set radially-outboard of the cavity 134 and of the shaft axis SA. In this embodiment, raised circular elevations 136 form and establish the grooves 132; still, in other embodiments the grooves could be formed and established in other ways. The grooves 132 receive insertion of the retention body/assembly 24 when the retention body/assembly 24 is fully deployed and engaged with the grooves 132. Upon engagement, the retention body/assembly 24 is retained within the confines of the particular groove 132 and able to move and ride along the groove 132 and within such confines. In the embodiment here, for instance, the retention body/assembly 24 can move and ride over the circular extent of the particular groove 132, while being precluded and prevented from other movements via the elevations 136. This capability serves to provide a guided articulated motion of the handle body 28 with respect to the frame body 30, which in turn provides corresponding guided articulated motion of the end effector body 14. Further, in this embodiment, when the retention body/assembly 24 is engaged with the cavity 134, a constraint in which the handle axis HA is retained in a coincident arrangement with the shaft axis SA is furnished. And when the retention body/assembly 24 is engaged with one of the grooves 132, a constraint in which the handle axis HA is retained in one of the multitude of misalignment arrangements with the shaft axis SA is furnished and guided articulated motions can be carried out.

Furthermore, in FIG. 13C, the retention holder 104 and retention domain 130 includes a multitude of retention structures 138 situated at a surface thereof. The retention structures 138 can take various forms in various embodiments. The retention structures 138 serve to provide retention capabilities with the retention body/assembly 24 upon their engagement. The retention body/assembly 24 can have retention structures at a distal end thereof that are similar and/or complementary to the retention structures 138 of the retention holder 104 and retention domain 130. The retention structures 138 can constitute a somewhat expansive portion and area at the rearside surface 110 (or other surface) of the second body/assembly 22 and frame body 30. In FIG. 13C, the retention structures 138 are in the form of a multitude of smaller-sized teeth structures 140 that project from the surface of the retention holder 104 and retention domain 130. The retention body/assembly 24 can have complementary teeth structures. Still, in other embodiments the retention structures could take other forms including hook-and-loop fasteners; and further still, other embodiments of the retention structures could take the form of numeral spherically-shaped cavities that complement a retention body/assembly in the form of a spring-loaded ball plunger. Upon engagement, the retention body/assembly 24 is retained with the retention holder 104 and retention domain 130 via the retention structures. Further, depending on the location of the retention body/assembly 24 with respect to the retention holder 104 and retention domain 130 upon their engagement, a constraint in which the handle axis HA is retained in a coincident arrangement with the shaft axis SA can be furnished, and a constraint in which the handle axis HA is retained in one of the multitude of misalignment arrangements with the shaft axis SA can be furnished.

Moreover, the retention body/assembly and retention holder can have other designs, constructions, components, and functionalities in other embodiments. In a further embodiment, a retention body/assembly could retain and constrain additional degrees of freedom provided in the retention assembly and/or in the handheld surgical instrument. A roll DoF and the unlimited roll functionality and capabilities at the end effector body could be retained and constrained from movement via the retention body/assembly or via an additional retention body/assembly. Further, in an embodiment, the control switch could control a retention function of a remote joint. In yet another embodiment, the retention body/assembly could be an electromechanical mechanism controlled by a distant switch or alternate way. Further, in an embodiment, the deactivation of the retention body/assembly could be dependent upon an alternate device state, such as a reprocessed state, a clean state, or a ready—for use state. Further, in an embodiment, the first body/assembly and second body/assembly need not necessarily be located and positioned adjacent to each other, and rather could be located and positioned at discrete remote locations; here, the retention body/assembly could have the form of a pair of intermediate bodies located and positioned at the first and second bodies/assemblies and connected to each other via a mechanical or electrical transmission member.

There are two notable functional impacts to the handheld surgical instrument when activation of the retention body/assembly occurs: prevent motions of the articulation input joint and introducing grounding joint degrees of constraint.

First, the restriction of pitch and yaw motions between the first body/assembly and second body/assembly prevents all input motions at the articulation input joint. The absence of articulation input joint motions preclude all motions of the articulation transmission members, preclude all articulation transmission member motions at the articulation output joint, and ultimately preclude all articulation motions of the output body. In other words, activation of the retention body/assembly precludes all motions of the output body locking it into a specific orientation.

Secondly, activation of the retention body/assembly introduces additional degrees of constraint to the grounding joint between the first body/assembly and the second body/assembly. Introduction of pitch degrees of constraint the yaw degrees of constraint between the first body/assembly and second body/assembly result in six degrees of constraint. This allows all rigid body user input motions at the first body/assembly to translate directly to the second body/assembly. Combined with the first functional impact of the articulation output motion when the retention body/assembly is activated, the rigid body motions of the second body/assembly then translate to the output body/assembly. In other words, the user may control rigid body motions of the output body/assembly by directly providing rigid body motions at the first body/assembly. The resulting configuration allows a surgeon to precisely control the position of the end effector body with a single hand when preparing to insert the handheld surgical instrument into a trocar during a surgical procedure.

From a user perspective the rigid body behaviors of the handheld surgical instrument differ depending on the position of the retention body/assembly. For instance, a user, typically a trained surgeon, manipulating the handheld surgical instrument by way of grasping the handle body at the user input with the retention body/assembly deactivated. With proper ergonomic form the surgeon positions their forearm in a generally horizontal orientation and raises their hand so that their wrist joint is in alignment with their forearm. While holding the handle body, the frame and shaft bodies of the device are free to drop and point toward the floor due to the degrees of freedom present between the handle and frame bodies. Contrastingly, the end effector articulation joint will articulate upward (i.e., pitch) due to the now articulated position of the articulation input joint (handle body rotated with respect to the frame body). This experience from a surgeon's point of view may be one of confusion. The surgeon may raise and lower the handle body or may articulate it back and forth resulting in seemingly indeterminate behavior. This behavior is due to having a single user input at the handle body and a device having multiple joints and bodies with degrees of freedom. More specifically, there are pitch and yaw articulation degrees of freedom between the handle body and frame body, there is an articulation transmission system that transmits handle articulation motions to the end effector body, and there are articulation degrees of freedom of the end effector body relative to the shaft body. To counter the indeterminate nature of the device and enable improved usability, there should be a second input that influences the behavior of the device. In fact, there are three unique inputs that aid usability in different scenarios: 1) shaft body support by surgeon or assistant, 2) shaft body support by trocar during surgical use, and 3) fully constraining the grounding joint by activation of the retention body/assembly.

1) In some situations, the surgeon may use their opposite hand to raise and support the shaft body of the device positioning it such that it is in alignment with their forearm and horizontal to the ground. Through this support of the shaft body the surgeon has full control of the rigid body motions of the frame and shaft body position and orientation. Furthermore, and while the surgeon continues to support the shaft body with one hand and grasps the handle body in the other, they begin to move their hand grasping the instrument handle body at the wrist joint. They may articulate their wrist joint in flexion (bend downward), extension (bend upward), abduction (bend toward the thumb), adduction (bend toward the little finger), or any combination thereof. In turn, the motions of the hand correlate directly to motions of the handle body. These motions are generally isolated from the frame body due to the degrees of freedom present between the first body/assembly and the second body/assembly and directly induce articulations of the end effector body that mimic the handle body motions. This allows the surgeon to precisely orient the end effector body as they wish. In total, the surgeon maintains full control of the device by means of grasping the handle body in one hand directing end effector body articulation motions and directing shaft rigid body motions with the opposite hand. As a practical matter, this use scenario occurs while the surgeon is holding the device in free space and in preparation for a procedure. This scenario culminates when the surgeon inserts the device end effector body into their patient via the trocar. The trocar, being a narrow and long cannula, requires the end effector body to be in axial alignment with the shaft body during insertion. Once inserted through the trocar, the use scenario shifts, and the trocar thereby becomes the second input type for the instrument.

2) The use scenario when the trocar serves as the additional input is the common configuration that allows the surgeon to control the instrument for its intended use. The trocar provides positional support for the shaft body of the device constraining two positional degrees of freedom while allowing the surgeon to control the remaining four degrees of freedom: move the instrument along the shaft axis inward toward the patient and outward by moving the handle body inward and outward, roll the device along the shaft axis by twisting or rolling the handle body along its handle axis, and control the orientation of the shaft body in the two remaining rotational degrees of freedom by shifting position of the device handle body. The surgeon controls the end effector body motion in this use scenario in the same manner as to when the shaft body is supported by the surgeon's opposite hand. In total, the surgeon maintains full control of the position and orientation of the end effector body.

3) The third additional input type is the subject of this disclosure and is an alternate method that addresses the drawbacks of the first method whereby the surgeon supports the shaft body with an opposite hand. Often, the opposite hand of the surgeon is not available to support the shaft body as they may be holding other instruments, thus presenting a challenge that should be addressed. Their grasp on the handle body allows them to pick up the device but does not enable them to accurately position the end effector body to be inserted into the long narrow cannula of the trocar. The shaft body support could come from a surgical assistant, but that also has drawbacks and introduces inefficiencies. The third additional input introduced is the actuation of the retention switch located on the instrument itself causing benefits of the functional impacts detailed above to be realized. The surgeon may control the rigid body motions of the device simply by controlling the orientation of the handle body while also maintaining an end effector body orientation that is locked in axial alignment with the shaft axis. The full instrument behaves as a single rigid body, allows the surgeon to precisely control and place the end effector body where they wish, and ultimately allows the surgeon to insert the end effector body into the cannula of the trocar with a one-handed motion. Once the instrument is supported by the trocar, the surgeon may deactivate the articulation retention and lockout feature by cycling a handle mounted switch to the OFF position and renew the degrees of freedom that allow the surgeon to use the instrument for its intended use.

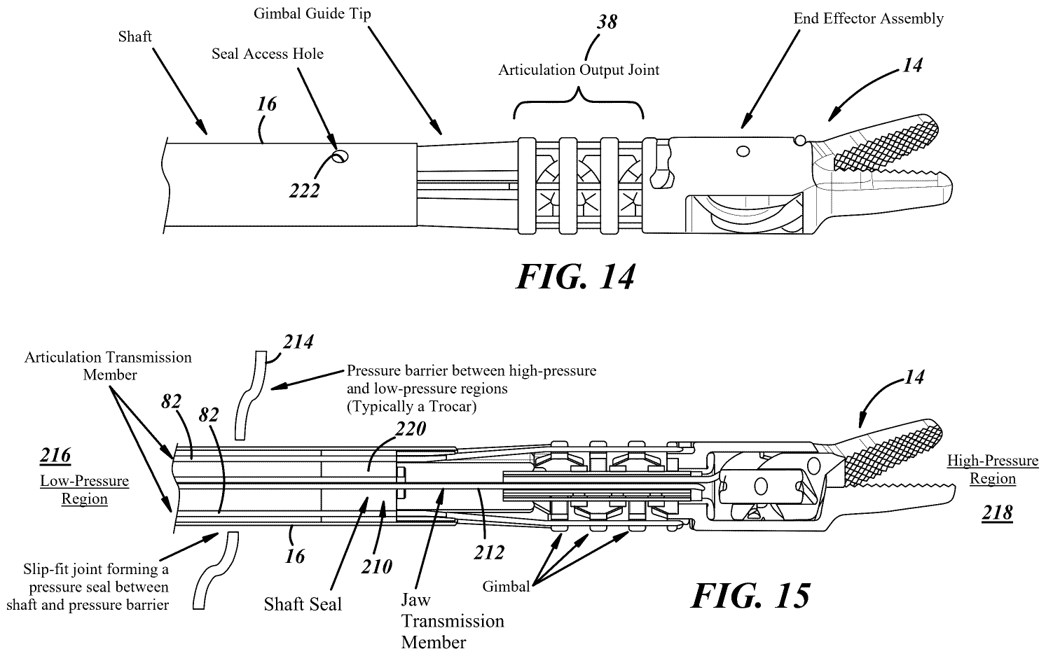

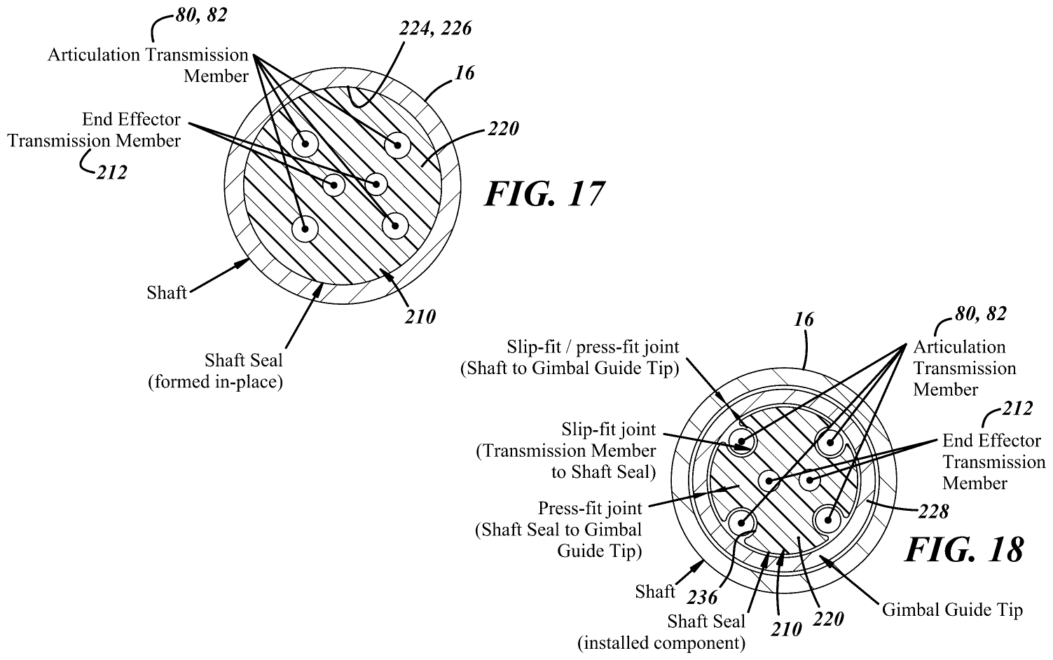

With reference now to FIGS. 14-18, embodiments of a shaft seal 210 are set forth that permit passage and movement of the yaw and pitch articulation transmission members 80, 82—as well as other transmission members such as end effector transmission members 212—while maintaining a pressure differential across a pressure barrier 214 residing adjacent the shaft seal 210.

In general, for use in minimally invasive surgery (MIS), instruments should maintain a pressure differential between the region in which the input body is located and the region in which the output body is located. A pressure differential should be maintained so that insufflation is maintained during a surgical procedure. A surgeon inserts a trocar into the patient and the shaft body 16 then passes through the trocar whereby a gas-tight seal is established between the outer diameter of the shaft body 16 and the trocar. As a result, the shaft body 16 spans a low-pressure region 216 and a high-pressure region 218 and therefore must contain some internal measures to isolate the pressure of the low- and high-pressure regions 216, 218 as well. Additionally, an internal shaft seal inhibits the transmission of fluids from the internal insufflated cavity to the user via the internal shaft channel. This disclosure presents two embodiments and methods to maintain the pressure differential within the shaft body 16.

In a first embodiment of FIGS. 14, 15, and 17, the shaft seal 210 includes a seal body 220. The shaft seal 210 and seal body 220 can be composed of a liquid silicone material, or a material with similar properties, that is curable and solidifies via the application of ultraviolet light thereto. In a liquid state, the liquid material is injected into the shaft body 16 via a seal access hole 222 residing in the shaft body 16 and spanning wholly therethrough. When injected, the liquid material fills an interior cavity 224 intended to receive the shaft seal 210 and seal body 220. The interior cavity 224 can be defined and established at an interior of the shaft body 16 and by a radial and axial region thereof. Once injected, the liquid material is cured and hardens in place by exposure to, and application of, ultraviolet light. The yaw and pitch articulation transmission members 80, 82 and the end effector transmission members 212 are immobilized amid curing and solidification of the liquid material and subsequent formation of the shaft seal 210 and seal body 220. In the first embodiment, the shaft seal 210 and seal body 220 are located at a distal end portion of the shaft body 16 and adjacent a proximal end portion of the articulation input joint 38 and end effector body 14.

Further, at the location where the shaft seal 210 and seal body 220 reside, the yaw and pitch articulation transmission members 80, 82 and end effector transmission members 212 also reside. Amid the curing and solidification process, a permanent bond is not formed between the shaft seal 210 and seal body 220 and the yaw and pitch articulation transmission members 80, 82 and end effector transmission members 212. Rather, the yaw and pitch articulation transmission members 80, 82 and end effector transmission members 212 are able to move with respect to the shaft seal 210 and seal body 220. Moreover, during the curing and solidification process, a suitable bond is formed between an internal surface 226 of the shaft body 16 and the shaft seal 210 and seal body 220, as well as between a proximal end of a gimbal guide tip and the shaft seal 210 and seal body 220, according to this embodiment. The bond formed here has been found to be sufficient to remain secured in place throughout the useful life of the larger handheld surgical instrument 12. While the shaft seal 210 and seal body 220 wholly envelope the yaw and pitch articulation transmission members 80, 82 and end effector transmission members 212, it has been found that such envelopment and intimate fit thereamong does not restrict motion of the transmission members or negatively affect system efficiencies in a material way during use of the handheld surgical instrument 12. Furthermore, the shaft seal 210 and seal body 220 furnishes a durable barrier that precludes and prevents insufflation pressure loss and/or movement of fluids during a surgical procedure. The shaft seal 210 and seal body 220 block insufflated gas from traveling along the shaft axis SA and though the shaft body interior and toward the low-pressure region 216.

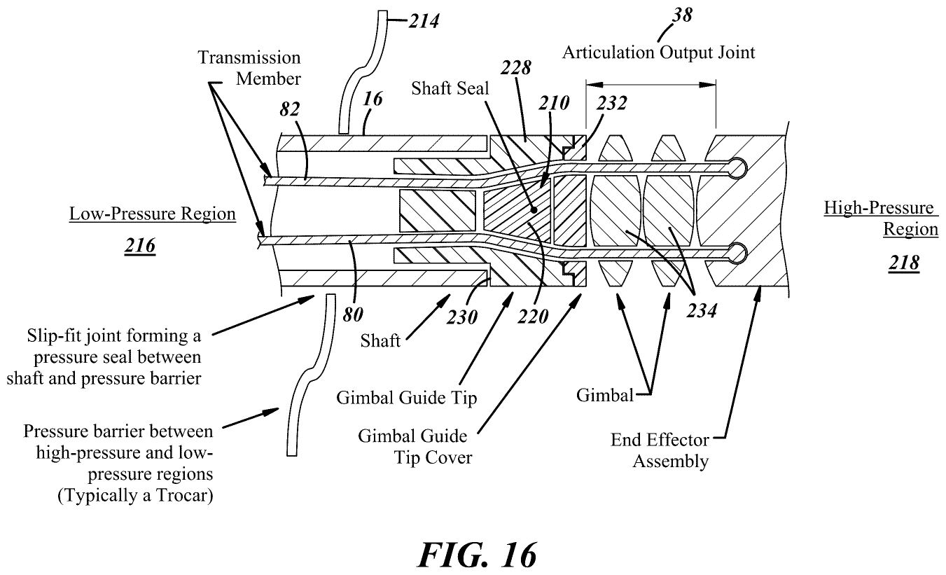

In a second embodiment of FIGS. 16 and 18, the shaft seal 210 and seal body 220 is an installed component. Here, the shaft seal 210 and seal body 220 can be composed of a low durometer, compressible material that is manufactured (e.g., injection molded) and installed in place during the assembly process of the handheld surgical instrument 12; still, a material with similar properties is possible. In the second embodiment, a gimbal guide tip body 228 is installed at the shaft body 16 with six DoC therebetween. The gimbal guide tip body 228 has a shoulder 230 that intimately fits within the shaft body 16 to prevent insufflation loss. An interface between the gimbal guide tip body 228 and the shaft body 16 may be a press-fit, slip-fit, contain a compressive component such as an o-ring, gasket, secured using an adhesive or epoxy, or be molded in place on the shaft body 16, among other possibilities. Further, a gimbal guide tip cover body 232 is secured at an end of the gimbal guide tip body 228. The gimbal guide tip cover body 232 seats with the gimbal guide tip body 228 in a similar way as described above for the gimbal guide tip body 228 and the shaft body 16. The gimbal guide tip cover body 232 provides a mating interface for gimbals 234 of the articulation output joint 38 whereby the gimbals 234 contain one (pitch) or two (pitch and yaw) degrees of freedom relative to the gimbal guide tip cover body 232. Furthermore, both of the gimbal guide tip body 228 and gimbal guide tip cover body 232 provide passage of the yaw and pitch articulation transmission members 80, 82 and end effector transmission members 212 via closely-fitting clearance holes or through-ways 236.

Further, in the second embodiment the shaft seal 210 and seal body 220 is located and positioned adjacent the gimbal guide tip body 228 and gimbal guide tip cover body 232, and radially-interiorly of the gimbal guide tip body 228, as depicted. The material composition of the shaft seal 210 and seal body 220 can possess a suitably low coefficient of friction with respect to the yaw and pitch articulation transmission members 80, 82 and end effector transmission members 212. The shaft seal 210 and seal body 220 are shaped and sized so that it exhibits a light press-fit (e.g., interference fit) to perimeter surfaces of the gimbal guide tip body 228. A seal is hence established thereat. The shaft seal 210 and seal body 220 has a slip fit joint to the yaw and pitch articulation transmission members 80, 82 and end effector transmission members 212, and is further sized for a tight fit and to minimize loss of insufflation pressure through any gaps. Moreover, in an alternative to this second embodiment, the shaft seal 210 and seal body 220 could be cured and solidified in place at its location shown adjacent the gimbal guide tip body 228 and gimbal guide tip cover body 232, as previously described in connection with the first embodiment, via the application of ultraviolet light thereto. Further, in yet another embodiment, the gimbal guide tip body 228 and seal body 220 could be combined as a single component composed of the same or different materials relative to each other.

Further, in general, the shaft seal 210 and seal body 220 can exhibit minimized impact on the motion of the yaw and pitch articulation transmission members 80, 82 and end effector transmission members 212, and should not substantially dampen tactile feedback that may otherwise be felt at the accompanying input body/assembly and first body/assembly 20 from the output body/assembly 26 and end effector body 14. Moreover, the shaft seal and seal body can have other designs, constructions, components, and functionalities in other embodiments. In an embodiment, the shaft seal and seal body could include a multitude of individual shaft seals and seal bodies-one for each of the transmission members; here, each of the shaft seal and seal body could be an o-ring, gasket, or annular ring diaphragm type sized to intimately mate with each transmission member. Further, in an embodiment, the shaft seal and seal body could accommodate for the passage of two-force members, drive shafts, control rods, rotating transmission members, electrical conductors, or other bodies that are used to transmit information, energy, or work. In an embodiment, the shaft seal and seal body could be composed of an elastomeric material such as liquid silicone rubber or a fluoropolomer such as PTFE. Yet further, in an embodiment, the shaft seal and seal body could provide a gas-tight seal around a cannula that allows the passage of objects or other fluids such as equipment necessary for biopsy, irrigation, or vision technologies.

The figures are not necessarily to scale, and some features may be exaggerated or minimized to show key details of components. Therefore, specific structural and functional details disclosed herein are not to be interpreted as limiting, but merely as a representative basis for teaching one skilled in the art to variously employ the present invention.

This disclosure is not intended to limit the scope to medical devices or the healthcare industry. For example, the system herein may be applied to conventional electro-mechanical control systems and industrial applications.

Furthermore, in general, while a multitude of embodiments have been depicted and described with a multitude of components in each embodiment, in alternative embodiments of the retention assembly the components of various embodiments could be intermixed, combined, and/or exchanged for one another. In other words, components described in connection with a particular embodiment are not necessarily exclusive to that particular embodiment.

As used herein, the terms “general” and “generally” and “substantially” are intended to account for the inherent degree of variance and imprecision that is often attributed to, and often accompanies, any design and manufacturing process, including engineering tolerances- and without deviation from the relevant functionality and intended outcome-such that mathematical precision and exactitude is not implied and, in some instances, is not possible. In other instances, the terms “general” and “generally” and “substantially” are intended to represent the inherent degree of uncertainty that is often attributed to any quantitative comparison, value, and measurement calculation, or other representation.

It is to be understood that the foregoing is a description of one or more aspects of the disclosure. The disclosure is not limited to the particular embodiment(s) disclosed herein, but rather is defined solely by the claims below. Furthermore, the statements contained in the foregoing description relate to particular embodiments and are not to be construed as limitations on the scope of the disclosure or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

Those of skill in the art will understand that modifications (additions and/or removals) of various components of the substances, formulations, apparatuses, methods, systems, and embodiments described herein may be made without departing from the full scope and spirit of the present disclosure, which encompass such modifications and any and all equivalents thereof.

1. A retention assembly, comprising:
a first body;
a second body;
a third body;
at least one joint situated between said first body and said second body, said at least one joint providing at least one degree of freedom between said first body and said second body; and
a retention body moveable between a first position and a second position;
wherein, when said retention body is in said second position, said retention body at least partially constrains at least one of said at least one degree of freedom between said first body and said second body, the at least partial constraint is further effected at said third body, and wherein, when said retention body is in said first position, the at least partial constraint between said first body and said second body via said retention body is absent, and the at least partial constraint at said third body is absent.

2. The retention assembly as set forth in claim 1, wherein said first body is a handle body and said second body is a frame body and said third body is an end effector body.

3. The retention assembly as set forth in claim 1, wherein said retention body is a pin body and the at least partial constraint is a full constraint of at least one of said at least one degree of freedom between said first body and said second body.

4. The retention assembly as set forth in claim 1, wherein, when said retention body is in said second position, said retention body engages with a retention domain at said second body.

5. The retention assembly as set forth in claim 1, wherein said retention body is moveable with respect to said first body and has a single degree of freedom with respect to said first body.

6. The retention assembly as set forth in claim 1, wherein said at least one degree of freedom includes a first rotational degree of freedom and a second rotational degree of freedom, and wherein, when said retention body is in said second position, said retention body concurrently at least partially constrains said first rotational degree of freedom and said second rotational degree of freedom.

7. The retention assembly as set forth in claim 1, wherein said third body is at least one intermediate body situated between said first body and said second body, said at least one joint including a first joint and a second joint, said first joint residing between said first body and said at least one intermediate body, and said second joint residing between said second body and said at least one intermediate body, said first joint providing a first rotational degree of freedom between said first body and said at least one intermediate body, and said second joint providing a second rotational degree of freedom between said second body and said at least one intermediate body, and wherein, when said retention body is in said second position, said retention body concurrently at least partially constrains said first rotational degree of freedom and said second rotational degree of freedom.

8. The retention assembly as set forth in claim 1, wherein said third body is an end effector body and, when said retention body is in said second position and said retention body at least partially constrains at least one of said at least one degree of freedom, movement of an end effector is correspondingly at least partially constrained.

9. The retention assembly as set forth in claim 1, wherein said first and second positions constitute bistable positional states of said retention body.

10. The retention assembly as set forth in claim 9, further comprising a detent mechanism that effects the bistable positional states of said retention body.

11. The retention assembly as set forth in claim 1, wherein said retention body has a plurality of positional states, said plurality of positional states including said first position and said second position and providing increasing degrees of at least partial constraint between said first body and said second body from said first position and to said second position.

12. The retention assembly as set forth in claim 1, wherein the retention assembly is a handheld surgical instrument retention assembly.

13. The retention assembly as set forth in claim 1, wherein the retention assembly is a robotic surgical instrument retention assembly.

14. The retention assembly as set forth in claim 1, wherein said retention body comprises at least one magnet body facilitating moveability from said first position and to said second position.

15. The retention assembly as set forth in claim 1, wherein moveability of said retention body between said first and second positions is via electric actuation.

16. The retention assembly as set forth in claim 1, wherein said third body has at least one rotational degree of freedom with respect to said first body, and wherein, when said retention body is in said second position, said retention body constrains said rotational degree of freedom between said first body and said third body, and wherein, when said retention body is in said first position, the constraint between said first body and said third body via said retention body is absent.

17. The retention assembly as set forth in claim 1, wherein said retention body is a lock body.

18. A retention assembly, comprising:
a first body;
a second body;
at least one joint situated between said first body and said second body, said at least one joint providing a first degree of freedom between said first body and said second body, and providing a second degree of freedom between said first body and said second body; and
a retention body having at least one positional state, said at least one positional state at least partially and concurrently constraining said first degree of freedom and said second degree of freedom between said first body and said second body, and said at least one positional state at least partially constraining movement of an end effector of said retention assembly.

19. The retention assembly as set forth in claim 18, further comprising an intermediate body, and wherein said at least one joint is situated among said first body, said second body, and said intermediate body, and said first and second degrees of freedom are among said first body, said second body, and said intermediate body.

20. The retention assembly as set forth in claim 19, wherein said at least one joint includes a first joint residing between said first body and said intermediate body and a second joint residing between said second body and said intermediate body, said first degree of freedom is provided at said first joint and is a first rotational degree of freedom between said first body and said intermediate body, and said second degree of freedom is provided at said second joint and is a second rotational degree of freedom between said second body and said intermediate body.

21. The retention assembly as set forth in claim 18, wherein said at least one positional state is a plurality of positional states that provide increasing degrees of the at least partial constraint of said first degree of freedom and said second degree of freedom.

22. The retention assembly as set forth in claim 21, wherein said plurality of positional states of said retention body comprises a first position, a second position, and a third position;
wherein, when said retention body is in said first position, said first and second degrees of freedom lack constraint via said retention body;
wherein, when said retention body is in said second position, said retention body partially and concurrently constrains said first and second degrees of freedom; and
wherein, when said retention body is in said third position, said retention body fully and concurrently constrains said first and second degrees of freedom.

23. The retention assembly as set forth in claim 18, wherein said at least one positional state is a first position and a second position, said first and second positions constituting bistable positional states of said retention body.

24. The retention assembly as set forth in claim 23, further comprising a detent mechanism that effects the bistable positional states of said retention body.

25. The retention assembly as set forth in claim 18, wherein said retention body is a single retention body and the at least partial and concurrent constraint of said first and second degrees of freedom is via said single retention body.

26. The retention assembly as set forth in claim 18, wherein the retention assembly is a handheld surgical instrument retention assembly.

27. The retention assembly as set forth in claim 18, wherein the retention assembly is a robotic surgical instrument retention assembly.

28. The retention assembly as set forth in claim 18, wherein said retention body comprises at least one magnet body facilitating moveability to said at least one positional state.

29. The retention assembly as set forth in claim 18, wherein moveability of said retention body to said at least one positional state is via electric actuation.

30. The retention assembly as set forth in claim 18, further comprising a third body having a rotational degree of freedom with respect to said first body, and wherein, when said retention body is in at least one of said at least one positional state, said retention body constrains said rotational degree of freedom between said first body and said third body.

31. A method of at least partially constraining at least one degree of freedom between bodies, the method comprising:
moving a retention body from a first position to a second position, wherein, when said retention body is in said second position, at least one degree of freedom between a first body and a second body is at least partially constrained, the at least partial constraint is further effected at a third body, and wherein, when said retention body is in said first position, the at least partial constraint of said at least one degree of freedom between said first body and said second body is absent, and the at least partial constraint at said third body is absent.

32. A retention assembly, comprising:
a first body;
a second body;
at least one joint situated between said first body and said second body, said at least one joint providing at least one degree of freedom between said first body and said second body; and
a magnet body carried by said first body or by said second body;
wherein, when said first body and said second body exhibit at least one position with respect to each other about said at least one joint, a magnetic field of said magnet body provides at least partial constraint at at least one of said at least one degree of freedom between said first body and said second body.

33. The retention assembly as set forth in claim 32, wherein said magnet body remains static with respect to said first body or said second body in which said magnet body is carried amid providing the at least partial constraint.

34. A handheld surgical instrument retention assembly, comprising:
a handle body;
a frame body coupled with said handle body;
an articulation input joint established between said handle body and said frame body, said articulation input joint providing a pitch rotational degree of freedom between said handle body and said frame body, and said articulation input joint providing a yaw rotational degree of freedom between said handle body and said frame body, movements of said handle body with respect to said frame body via said articulation input joint and about said pitch and yaw rotational degrees of freedom furnishes corresponding movements at at least one end effector of the handheld surgical instrument;
at least one retention body carried by said handle body or carried by said frame body, said at least one retention body moveable with respect to said handle body or said frame body at least between a retracted position and a deployed position; and
at least one retention holder located at the other of said handle body or said frame body;
wherein, upon movement of said at least one retention body to said deployed position and engagement of said at least one retention body with said at least one retention holder, movements of said handle body with respect to said frame body via said articulation input joint and about said pitch rotational degree of freedom and about said yaw rotational degree of freedom are at least partially constrained and corresponding movements of the at least one end effector are at least partially constrained.

35. The handheld surgical instrument retention assembly as set forth in claim 34, wherein said handle body establishes a handle axis over an elongated extent of said handle body and said frame body has a shaft body and said shaft body establishes a shaft axis over an elongated extent of said shaft body, and wherein, upon at least partial constraint of movements of said handle body with respect to said frame body, said handle axis is retained in a coincident arrangement with respect to said shaft axis or in one of a plurality of misalignment arrangements with respect to said shaft axis.

36. The handheld surgical instrument retention assembly as set forth in claim 34, wherein said handle body establishes a handle axis over an elongated extent of said handle body and said frame body has a shaft body and said shaft body establishes a shaft axis over an elongated extent of said shaft body, and wherein the at least partial constraint is full constraint and, upon full constraint of movements of said handle body with respect to said frame body, said handle axis is retained in a coincident arrangement with respect to said shaft axis.

37. The handheld surgical instrument retention assembly as set forth in claim 34, wherein said handle body establishes a handle axis over an elongated extent of said handle body and said frame body has a shaft body and said shaft body establishes a shaft axis over an elongated extent of said shaft body, and wherein the at least partial constraint is full constraint and, upon full constraint of movements of said handle body with respect to said frame body, said handle axis is retained in one of a plurality of misalignment arrangements with respect to said shaft axis.

38. The handheld surgical instrument retention assembly as set forth in claim 34, wherein said retention body is a pin body carried by said handle body and moveable with respect to said handle body between said retracted position and said deployed position, and said retention holder is a cavity residing in said frame, said pin body is inserted in said cavity upon their engagement.

39. The handheld surgical instrument retention assembly as set forth in claim 34, wherein said handle body establishes a handle axis over an elongated extent of said handle body and said frame body has a shaft body and said shaft body establishes a shaft axis over an elongated extent of said shaft body, and wherein said retention holder comprises a retention domain with a plurality of retention structures situated at a surface of said retention holder, said retention domain and said plurality of retention structures exhibiting general confrontation with said retention body, and wherein, upon movement of said retention body to said deployed position and engagement of said retention body with said retention domain and with said plurality of retention structures, said handle axis is retained in one of a plurality of misalignment arrangements with respect to said shaft axis.

40. The handheld surgical instrument retention assembly as set forth in claim 39, wherein said plurality of retention structures comprises a plurality of teeth structures projecting from said surface of said retention holder at said retention domain.

41. The handheld surgical instrument retention assembly as set forth in claim 39, wherein said handle body establishes a handle axis over an elongated extent of said handle body and said frame body has a shaft body and said shaft body establishes a shaft axis over an elongated extent of said shaft body, and wherein said retention body comprises a plurality of second retention structures, and wherein, upon engagement of said plurality of retention structures of said retention holder with said plurality of second retention structures of said retention body, said retention body is retained with said retention holder and said handle axis is retained in one of the plurality of misalignment arrangements with respect to said shaft axis.

42. The handheld surgical instrument retention assembly as set forth in claim 34, wherein said retention body is carried by said handle body and said retention holder is carried by said frame body, and said retention holder comprises at least one groove that receives said retention body upon movement of said retention body to said deployed position and engagement of said retention body with said at least one groove, and wherein, upon engagement of said retention body with said at least one groove, said retention body is retained within said at least one groove and rideable along said at least one groove upon movement of said handle body therealong with respect to said frame body.

43. The handheld surgical instrument retention assembly as set forth in claim 42, wherein said handle body establishes a handle axis over an elongated extent of said handle body and said frame body has a shaft body and said shaft body establishes a shaft axis over an elongated extent of said shaft body, and wherein said at least one groove comprises at least one circular groove having a coincident arrangement with respect to said shaft axis, and wherein, upon movement of said handle body along said at least one circular groove and riding of said retention body along said at least one circular groove, said at least one circular groove provides guided articulated motion of said handle body with respect to said frame body and with said handle axis in misalignment arrangements with respect to said shaft axis and with corresponding articulated movements at the end effector of the handheld surgical instrument.

44. The handheld surgical instrument retention assembly as set forth in claim 34, further comprising a deviation ring body situated between said handle body and said frame body, said articulation input joint established at said deviation ring body and said pitch and yaw rotational degrees of freedom provided about said deviation ring body.

45. The handheld surgical instrument retention assembly as set forth in claim 44, further comprising a first half-ring body extending from said handle body to said deviation ring body, and comprising a second half-ring body extending from said frame body to said deviation ring body.

46. The handheld surgical instrument retention assembly as set forth in claim 34, further comprising an intermediate body situated between said handle body and said frame body, and wherein said at least one retention body includes a first retention body and a second retention body, said at least one retention holder includes a first retention holder and a second retention holder, and the at least one effector includes a first end effector and a second end effector, and wherein, upon movement of said first and second retention bodies to said deployed positions and engagement of said first and second retention bodies with said respective first and second retention holders, movements of said handle body with respect to said frame body via said articulation input joint and about said pitch rotational degree of freedom and about said yaw rotational degree of freedom are at least partially constrained and corresponding movements of the first and second end effectors are at least partially constrained.
